(19) United States
(12) Patent Application Publication
(10) Pub. No.: US 2002/0061839 A1
(43) Pub. Date: May 23, 2002

(54) Serine peptidase modulators

(76) Inventors: Simon Lodewijk Scharpe, Antwerpen (BE); Ingrid Anna Jozef De Meester, Wilrijk (BE); Anne-Marie Virginie Renee Lambeir, Heverlee (BE); Koen Jan Ludovicus Augustyns, Minderhout (BE); Achiel Jean-Marie Haemers, Sint-Martens-Latem (BE); Filip Jozef Anny Goossens, Kampenhout (BE); Dirk Frans Hendriks, Aartselaar (BE); Alexandre Arkadicvitch Beliaev, Mindelo-Vila do Conde (PT)

Correspondence Address: Barbara E. Johnson, WEBB ZIESENHEIM LOGSDON ORKIN & HANSON, P.C., 700 Kopppers Building, 436 Seventh Avenue, Pittsburgh, PA, 15219-1818, US

(21) Appl. No.: 09/837,537

(22) Filed: Apr. 18, 2001

Related U.S. Application Data
(63) Continuation of application No. 09/623,752, filed on Nov. 16, 2000.
Application No. 09/623,752 is a 371 of international application No. PCT/EP99/01617, filed on Mar. 9, 1999.

(30) Foreign Application Priority Data
Mar. 9, 1998 (EP) 98200733.8

(51) Int. Cl. A61K 38/16; A01N 1/00; A01N 1/02; C07K 5/00; C07K 7/00; C07K 16/00; C07K 17/00; A61K 38/04; A61K 38/00; A01N 65/00; A01N 63/00

(52) U.S. Cl. 514/7; 530/330; 514/17; 435/1.1; 424/93.1

(57) ABSTRACT

The present invention relates to new compounds having modulatory (inhibitory and stimulatory) activity on serine peptidases and proteases in general and dipeptidyl peptidase IV, prolyl oligopeptidase (PO), dipeptidyl peptidase II (DPP II), fibroblast activation protein α (FAPα), lysosomal Pro—X carboxypeptidase and elastase in particular. These new compounds can be used for the treatment of a variety of disease states in which these peptidases are involved.

### FIELD OF THE INVENTION

The present invention relates to novel modulators (inhibitors and stimulators) of serine peptidases and proteases in general and dipeptidyl peptidase IV, prolyl oligopeptidase (PO), dipeptidyl peptidase II (DPP II), fibroblast activation protein a (FAPα), lysosomal Pro—X carboxypeptidase and elastase in particular. The invention further relates to the preparation and use of these compounds for selective modulation (inhibition or stimulation) of serine peptidases and proteases and to pharmaceutical preparations comprising them. The terms “peptidase” and “protease” are used interchangeably.

### BACKGROUND OF THE INVENTION

Serine peptidases/proteases, like granzymes, mast cell tryptase, elastases, trypsin-like enzymes, prolyl oligopeptidase, dipeptidyl peptidase II and dipeptidyl peptidase IV are involved in various processes that take place in the body, such as blood coagulation, inflammation, immune response, and control of peptide hormone metabolism in general. Although serine peptidases are a physiological necessity they may also constitute a potential health hazard in case serine peptidase activity in the body is not controlled.

Serine peptidases have been described to be involved in various medical indications. Blood coagulation serine proteases are for example responsible for vascular clotting as well as cerebral and coronary infarction. Chymotrypsin-like enzymes and plasmin are involved in tumour invasion, tissue remodeling and clot dissociation. Pancreatitis, emphysema, rheumatoid arthritis, inflammation and adult respiratory distress syndrome may in some instances be caused by the uncontrolled proteolysis by other serine proteases such as elastase.

Serine peptidases form a large group with many members that are divided into clans and families. One member of the clan SC is dipeptidyl peptidase IV (DPP IV, EC 3.4.14.5), which is a highly specific exopeptidase with a serine type mechanism of protease activity, cleaving off dipeptides from the amino-terminus of peptides with proline or alanine at the penultimate position. In addition the slow release of dipeptides of the type X—Gly or X—Ser is reported for some naturally occurring peptides. DPP IV is constitutively expressed on epithelial and endothelial cells of a variety of different tissues, and is also found in body fluids. In the hematopoietic system, DPP IV was identified as the leukocyte antigen CD26.

Prolyl oligopeptidase (PO, EC 3.4.21.26) was discovered in the human uterus as an oxytocin-degrading enzyme. The enzyme shows a high specificity for proline residues and hydrolyses the peptide bond at its carboxyl side, provided the proline is not at the peptide amino-terminus. This endopeptidase has like DPPIV, a serine type mechanism and it is characterised by its activity on oligopeptides. PO cleaves specifically the Pro—Xaa bond in biological active peptides (substance P, ocytoxin, vasopressin, gonadoliberin, bradykinin, neurotensin) and it is likely to participate in the in vivo regulation of their actions. A role for PO in memory and other neural processes has been proposed (Yoshimoto T. and Ito K. in Handbook of proteolytic enzymes, eds. Barrett et al., Academic Press, 1998, p. 272-374).

Fibroblast activation protein α (FAPα) was discovered as a cell surface antigen of cultured normal fibroblasts. Its expression in vivo revealed to be very restricted on normal cells. In contrast, activated tumor stromal fibroblasts found in certain carcinomas express high levels of FAPα. The biological role of FAPα expression remains to be elucidated but speculations on functions in tissue remodeling and repair have been made (Rettig, FAPα in Barrett supra, p. 385-389.).

Dipeptidyl peptidase II (DPPII, EC 3.4.14.2) releases N-terminal dipeptides from oligopeptides, provided their N-termini are unsubstituted. The preferred P1 residues are Ala and Pro. An increase in serum DPPII has been observed in cancer patients and extremely high levels of DPPII are present in human carcinoma cells. OPPII can be inhibited by the classical (unspecific) inhibitors of serine type peptidases (J. K. McDonald in Barrett, supra, p. 408-411).

Elastases are defined by their ability to release soluble peptides from insoluble elastin fibers by a proteolytic process called elastinolysis. Elastase belongs to the chymotrypsin family of leucocyte serine-type proteases. Human leucocyte elastase (EC 3.4.21.37) preferentially cleaves peptides with a Val in P1 but also peptide bonds with Ala, Ser and Cys in P1 are hydrolyzed and it is believed to possess an extended substrate-binding site. The possible involvement of leucocyte elastase in inflammatory diseases, triggered the search for development of specific inhibitors. Moreover, a pathological role in lung emphysema, cystic fibrosis and adult respiratory distress syndrome has been suggested (J. Bieth in Barrett, supra, p. 54-60; D. Farley et al. in Pharmaceutical Enzymes, ed. A. Lauwers and S. Scharpé, Marcel Dekker, Inc., 1997, p. 306-326).

Lysosomal Pro—X carboxypeptidase (prolylcarboxypeptidase, angiotensinase C, EC 3.4.16.2) cleaves C-terminal amino acids from peptides with the general structure X—Pro—Y, where X is either a blocking group, another protected amino acid, or a peptide, and Y is an aromatic or aliphatic amino acid with a free carboxylic group. The enzyme is recovered from the lysosomal fraction of different tissues. Although the enzyme has an acidic pH optimum for small synthetic substrates (pH 5.0), it retains 50% of its maximal activity at physiological pH towards larger peptide substrates. (Des-Arg9)-bradykinin and angiotensin II are possible natural substrates for lysosomal Pro—X carboxypeptidase (Tan, F. and Erdös, E. in Barrett et al., supra, p. 405-407)

Because of their role in various physiological processes it is desirable to interfere in the activity of serine peptidases. Such interference can be either stimulation or inhibition. Various types of serine peptidase inhibitors have been described in for example EP-764 151 of the present inventors. The application inter alia describes compounds of the general formula Z—Xaa—Y′ wherein Z may or may not be present and is a protecting group, Xaa represents a dipeptide or an amino acid and Y′ may be a phosphonate, such as a diphenyl phosphonate. Further research of the present inventors has revealed that the toxicity thereof is not yet acceptable and the potency/efficacy are not sufficient.

U.S. Pat. No. 5,543,396 of Powers et al. relates to proline phosphonate derivatives. The phosphonate may be substituted with one or two phenyl groups which in turn may be mono-, di- or trisubstituted with a halogen, C₁-C₆ alkyl, C₁-C₆ perfluoralkyl, C₁-C₆ alkoxy, NO₂, CN, OH, CO₂H, amino, C₁-C₆ alkylamino, C₂-C₁₂ dialkylamino, C₁-C₆ acyl, and C₁-C₆ alkoxy-CO—, C₁-C₆ alkyl-S—. The present inventors, in the research that led to this invention, developed independently the same compounds. However, they found that regarding toxicity, stability and efficacy these compounds did not perform optimally.

It is therefore the object of the present invention to provide inhibitors of serine peptidases/proteases that have a more optimal combination of inhibitor capacity, stability in plasma, safety, bioavailability, duration of action and straightforward synthesis. In addition, the invention has for its object to provide compounds that have a stimulating activity on serine peptidases/proteases. These two types of compounds of the invention will also be identified herein as “modulating compounds”¹. More in particular, the invention provides compounds having such a more optimal combination for modulating the activity of DPP IV, PO, DPP II, FAPα, lysosomal Pro—X carboxypeptidase and elastase.

Thus, in the research that led to the present invention, the influence of different functional groups on the inhibitory (or stimulatory) activity of phosphonates was investigated. Prolylpyrrolidine diphenyl phosphonates were synthesized, substituted on the phenyls with hydroxyl, methoxy, acylamino, sulfonylamino, ureyl, methoxycarbonyl and alkylaminocarbonyl groups. The phenylesters were also replaced by other groups with good leaving group capacities such as trichloroethyl and trifluoroethyl. The inhibitory activity in vitro and in vivo on DPP IV and other serine peptidases, the stability and specificity of these compounds was tested.

It was then found that compounds as claimed in claim 1 are very potent modulators, in particular inhibitors, of serine peptidases/proteases in general and DPP IV, DPP II, PO, FAPα, lysosomal Pro—X carboxy-peptidase and elastase in particular. The compounds as listed in claim 12 were found to be potent inhibitors of DPP IV and PO.

The compounds of the invention are based on peptides. These peptides are constituted by either naturally occurring amino acids or other amino acids. The C-terminal carboxyl function is replaced by a phosphonate group.

The compounds of the invention are represented by the general formula I:

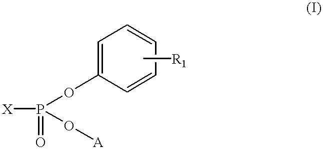

wherein

A is - - - (R2) or H or C₁-C₆ alkyl or halogenoalkyl, except perfluoroalkyl,

the phenyl group is mono-, di- or trisubstituted with R1 or R2;

X is a peptide- or amino acid-derived moiety;

A and the phenyl group substituted with R1 may optionally form a biphenyl diester;

all R1 substituents and R2 substituents are each independently selected from the group consisting of:

a) C₁-C₆ acylamino;

b) aroylamino, optionally substituted at the o- and/or p- and/or m-position with alkyl, in particular C₁-C₆ alkyl, and/or a halogen;

c) C₁-C₆ alkylsufonylamino;

d) arylsulfonylamino, optionally substituted at the o- and/or p- and/or m-position with alkyl, in particular C₁-C₆ alkyl, and/or a halogen;

e) α aminoacylamino wherein the α aminoacyl represents a side chain blocked or unblocked α-amino acid residue with the L, D or DL configuration at the α-carbon atom selected from the group consisting of:

alanine, methionine, methionine sulfoxide, arginine, homoarginine, phenylalanine, aspartic acid, proline, hydroxyproline, asparagine, serine, cysteine, threonine, histidine, glycinie, tyrosine, glutamic acid, pyroglutamic acid, tryptophan, glutamine, valine, norvaline, isoleucine, lysine, leucine, norleucine, thioproline, homoproline, 1,2,3,4-tetrahydro-isoquinoline-3-carboxylic acid (Tic), 2,3-dihydroindol-2-carboxylic acid, α-naphtylglycine, α-phenylglycine, 4-amidinophenylglycine, 4-phenylproline, 4-amidinophenylalanine, O-benzyl tyrosine, omega-acetyl lysine, α-aminobutyric acid, citrulline, homocitrulline, ornithine, o-methylserine, o-ethylserine, S-methylcysteine, S-ethylcysteine, S-benzylcysteine, homoserine, 4-dehydroproline, penicillamine, β-(2-thienyl)alanine, NH₂—CH(CH₂CHEt₂)—COOH, α-aminoheptanoic acid, NH₂—CH(CH₂-1-naphthyl)—COOH, NH₂—CH(CH₂-2-naphthyl)—COOH, NH₂—CH(CH₂-cyclohexyl)—COOH, NH₂—CH[CH₂-cyclohexyl)₂]—COOH, NH₂—CH(CH₂-cyclopentyl)—COOH, NH₂—CH[CH—(cyclopentyl)₂]—COOH, NH₂—CH (CH₂-cyclobutyl)—COOH, NH₂—CH[CH—(cyclobutyl)₂]—COOH, NH₂—CH(CH₂—cyclopropyl)—COOH, NH₂—CH[CH—(cyclopropyl)₂]-COOH, 5,5,5-trifluoroleucine, hexafluoroleucine, (S)-azetidine-2-carboxylic acid, (S)-pipecolic acid, (S)-oxazolidine-4-carboxylic acid, (R)-thiazolidine-4-carboxylacid (L-thioproline), sarcosine;

f) residue selected from the group consisting of 3-aminobenzoic acid, ε-aminocaproic acid, β-alanine;

g) Y—NH—CO—NH—;

h) Y′O₂CCH(NHCO—Y)—CH₂—;

i) Y′NHCO—;

j) CH₃—O—CO—Y′—NH—CO—;

k) CH₃—CH₂—O—CO—Y′—NH—CO—;

wherein Y is C₁-C₆ alkyl, aryl or H and Y′ is C₁-C₆ alkyl,

and pharmaceutically acceptable salts thereof.

In a specific embodiment of the invention X is a moiety of the general formula (AA)ₚ-aa—,

wherein:

p indicates that there may be 0, 1, 2, 3, 4 or 5 residues AA, which can be the same or different within one molecule;

AA and aa are selected from one of the following:

a) α-amino carboxylic acids with in α position an optionally substituted C₁-C₆ alkyl or aryl or aralkylmoiety;

b) alanine, methionine, methionine sulfoxide, arginine, homoarginine, phenylalanine, aspartic acid, proline, hydroxyproline, asparagine, serine, cysteine, threonine, histidine, glycine, tyrosine, glutamic acid, pyroglutamic acid, tryptophan, glutamine, valine, norvaline, isoleucine, lysine, leucine, norleucine, thioproline, homoproline, 1,2,3,4-tetrahydro-isoquinoline-3-carboxylic acid (Tic), 2,3-dihydroindol-2-carboxylic acid, α-naphtylglycine, α-phenylglycine, 4-amidinophenylglycine, 4-phenylproline, 4-amidinophenylalanine, O-benzyl tyrosine, omega-acetyl lysine, α-aminobutyric acid, citrulline, homocitrulline, ornithine, o-methylserine, o-ethylserine, S-methylcysteine, S-ethylcysteine, S-benzylcysteine, homoserine, 4-dehydroproline, penicillamine, β-(2-thienyl) alanine, NH₂—CH(CH₂CHEt₂)—COOH, α-aminoheptanoic acid, NH₂—CH(CH₂-1-naphthyl)—COOH, NH₂—CH(CH₂-2-naphthyl)—COOH, NH₂—CH(CH₂—cyclohexyl)—COOH, NH₂—CH[CH—(cyclohexyl)₂]—COOH, NH₂—CH(CH₂-cyclopentyl)—COOH, NH₂—CH[CH—(cyclopentyl)₂]—COOH, NH₂—CH(CH₂-cyclobutyl)—COOH, NH₂—CH[CH—(cyclobutyl)₂]—COOH, NH₂—CH(CH₂—cyclopropyl)—COOH, NH₂—CH[CH—(cyclopropyl)₂]—COOH, 5,5,5-trifluoroleucine, hexafluoroleucine, (S)-azetidine-2-carboxylic acid, (S)-pipecolic acid, (S)-oxazolidine-4-carboxylic acid, (R)-thiazolidine-4-carboxylacid (L-thioproline), 3-aminobenzoic acid, sarcosine, ε-aminocaproic acid, β-alanine,

wherein the alpha amino residue may be side chain blocked or unblocked and has the L, D, or DL configuration at the alpha carbon atom;

and pharmaceutically acceptable salts thereof.

In an alternative embodiment of the invention X is M—(AA)ₚ—aa—

wherein:

p, AA and aa are as defined above; and

M is selected from:

a) the group consisting of optionally substituted —CONH₂, —CSNH₂, —SO₂NH₂, phenyl-SO₂—, phenyl-CH₂SO₂—, 2-furyl-acryloyl; and

b) the group of protecting groups consisting of:

acetyl, adamantyloxycarbonyl, benzyloxycarbonyl, benzoyl, benzyl, t-butoxycarbonyl, t-butyl, 2,4-dinitrophenyl, formyl, fluorenylmethoxycarbonyl, 4-methoxybenzyl, tosyl, trifluoroacetyl, trityl, phthaloyl, phenylalkylcarbonyl, 2-indanylacetyl, 2-(1,2,3,4-tetrahydronaphtyl) acetyl, 4-(4-benzylphenoxy)alkyl;

In a specific embodiment x represents AA-aa-, wherein aa is proline and AA is as defined above. In an alternative embodiment X represents AA-aa-, wherein AA and aa are both proline. In case aa is alanine, R1 and R2 may further be selected from:

l) H, halogen, NO₂₁ CN, OH, COOH

m) amino, C₁-C₆ alkylamino, C₂-C₁₂ dialkylamino,

n) C₁-C₆ acyl

o) C₁-C₆ alkoxy-CO—

p) C₁-C₆ alkyl-S—.

In such compounds aa is alanine and preferably at least the AA coupled to aa is proline or phenylalanine. Preferred examples of the compounds are Phe—Ala-diphenylphosphonate or Pro—Ala-diphenylphosphonate and pharmaceutically acceptable salts thereof.

The invention can be divided in three groups of compounds.

In preferred compounds of this invention the phosphonate group is a diphenyl-phosphonate group (indicated with the symbol P(OPh)2), which is preferably substituted. The first group thus consists of compounds of the general formula II:

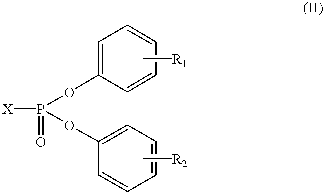

X—

O

wherein the substituents R1, R2 and X are as defined above (group 1).

Alternatively, the compounds are 2,2′ biphenyl diesters of α-aminoalkyl phosphonic acid having the general formula III:

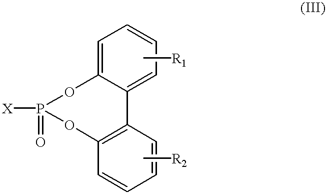

wherein the substituents R1, R2 and X are as defined above (group 2).

The third group (group 3) consists of compounds having the general formula IV:

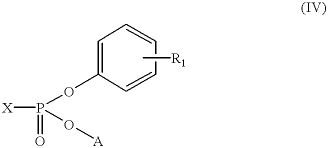

wherein X and R1 are as defined above, A is H or C₁-C₆ alkyl or halogenoalkyl, except perfluoroalkyl.

Specific members of these three groups will be listed below.

For inhibition of the serine protease cathepsin G, X is preferably selected from Cbz—Gly—Leu—Phe—, Z—Phe—Pro—Phe, and Suc—Val—Pro—Phe—. For prolyl oligopeptidase X may be selected from among the following: Cbz—Gly—Gly—Pro—, Cbz—Pro—Pro—, Boc—Val—Pro—Val—, MeO—Suc—Ala—Ala—Ala—Val—, MeO—Suc—Ala—Ala—Pro—Val—. For DPP IV, X is as follows: Ala—Pro—, Pro—Pro—, Ala—Pip—, Phe—Pro—, Ile—Pro—, Arg—Pro—, pF—Phe—Pro—, cyclohexylala-Pro—, Pro-azetidine—, Phe-azetidine—, Lys—Pro—, Lys—azetidine—. X is selected from among Suc—Lys(Cbz)—Val—Pro—Val—, Z—Ala—Ala—Ala and Boc—Val—Pro—Val— in case the enzyme to be inhibited is human leukocyte elastase. For Granzyme A, X may be selected from Cbz-(4-amidinophenylalanine)—, Z—Met—, 3-phenyl propanoyl-Pro-(4-aminophenylalanine)—, Cbz—Thr-(4-amidinophenylglycine)—, and Boc—D—Phe—Pro—(4-amidinophenylalanine)—. X may be Z—Phe—Pro—Phe—, Z—Phe—, Suc—Val—Pro—Phe—, MeO—Suc—Ala—Ala—Pro—Phe—, or MeO—Suc—Ala—Ala—Ala—Phe— when the enzyme to be inhibited is chymotrypsine. For trypsin-like serine-type proteases, X may be selected from among Cbz—Orn—, Cbz—Lys—Ala—, Cbz—Lys, Cbz—HomoLys—, Cbz—(4-amidinophenylalanine)—, Cbz-(4-amidinophenylglycine)—, Ph—CH₂—SO₂—Gly—Pro—(4-amidino-phenylglycine)—, 3-(2-furyl)acryloyl-(4-amidinophenylglycine), Cbz—Lys—(4-amidinophenylglycine)—, Cbz—Lys—Ala-(4-amidinophenylglycine)—, Cbz—Thr—(4-amidinophenylglycine)—, 3-(2-furyl)acryloyl-(4-amidinophenylalanine)—, Cbz—Ala—(4-amidinophenylglycine)—, Cbz—Ala—Ala—Ala—(4-amidinophenylglycine)—, 2-phenoxybenzoyl-Pro—(4-amidinophenylglycine)—, 3-phenoxybenzoyl-Pro—(4-amidinophenylglycine)—, 3-phenyl propanoyl-Pro—(4-amidinophenylalanine)—, 3,3-diphenyl propanoyl-Pro—(4-amidinophenylglycine)—. For inhibition of V8 protease of S. aureus X can be either Acetyl.Glu— or Acetyl.Asp—.

R1 and R2 are preferably selected from the group consisting of: 3-AcNH, 4-AcNH, 4-MeSO₂NH, 3-H₂NCONH, 3-H₂NCONH, 4-(N—Bz—Gly—NH), 4-(H—Gly—NH), 4(H—(S)—Ala—NH), 4-((S)—Pyr—NH), 4-((2S)—MeO₂CCH(NHAc)CH₂), 4-MeO₂C, 4-(EtO₂CCH₂NHCO), 4-(MeO₂C(CH₂)₂NHCO), 4-CH₃(CH₂)₂NHCO

Particularly preferred compounds of group 1 (formula II) are the following:

Di(3-acetamidophenyl) 1-(benzyloxycarbonyl-(S)-prolyl)pyrrolidine-2(R,S)-phosphonate (10d);

Di(4-acetamidophenyl) 1-(benzyloxycarbonyl-(S)-prolyl)pyrrolidine-2(R,S)-phosphonate (10e);

Di(⁴-methylsulfonylaminophenyl) 1-(benzyloxycarbonyl-(S)-prolyl)pyrrolidine-2(R,S)-phosphonate (10f);

Di(3-ureylphenyl) 1-(benzyloxycarbonyl-(S)-prolyl)pyrrolidine-2(R,S)-phosphonate (10g);

Di[4-(N-benzoylglycylamino)phenyl]-1-(benzyloxycarbonyl-(S)-prolyl)pyrrolidine-2(R,S)-phosphonate (10h);

Di[4-(N-benzyloxycarbonylglycylamino)phenyl]-1-(benzyloxycarbonyl-(S)-prolyl)pyrrolidine-2(R,S)-phosphonate (10i);

Di[4-(N-benzyloxycarbonyl-(S)-alanylamino)phenyl]-1-(benzyloxycarbonyl-(S)-prolyl)pyrrolidine-2(R,S)-phosphonate (10j);

Di[4-((S)-pyroglutamylamino)phenyl]-1-(benzyloxycarbonyl-(S)-prolyl)pyrrolidine-2(R,S)-phosphonate (10k);

Di{4-[-(S)-(2-methoxycarbonyl-2-acetamido)ethyl]-phenyl}1-(benzyloxycarbonyl-(S)-prolyl)pyrrolidine-2(R,S)-phosphonate (10l);

Di(4-methoxycarbonylphenyl)1-(tert-butyloxycarbonyl-(S)-prolyl)pyrrolidine-2(R,S)-phosphonate (10m);

Di{4-[(ethoxycarbonyl)methylaminocarbonyl]phenyl}1-(benzyloxycarbonyl-(S)-prolyl)-pyrrolidine-2(R,S)-phosphonate (10n);

Di{4-[2-(methoxycarbonyl)ethylaminocarbonyl]phenyl}1-(benzyloxycarbonyl-(S)-prolyl)-pyrrolidine-2(R,S)-phosphonate (10o);

Di[4-(n-propylaminocarbonyl)phenyl]1-(benzyloxycarbonyl-(S)-prolyl)-pyrrolidine-2(R,S)-phosphonate (10p);

Di(3-acetamidophenyl) 1-((S)-prolyl)pyrrolidine-2(R,S)-phosphonate hydrochloride (11d);

Di(4-acetamidophenyl) 1-((S)-prolyl)pyrrolidine-2(R,S)-phosphonate hydrochloride (11e);

Di(4-methylsulfonylaminophenyl) 1-((S)-prolyl)-pyrrolidine-2(R,S)-phosphonate hydrochloride (11f);

Di(3-ureylphenyl) 1-((S)-prolyl)pyrrolidine-2(R,S)-phosphonate hydrochloride (11g);

Di[4-(N-benzoylglycylamino)phenyl]-1-((S)-prolyl)-pyrrolidine-2(R,S)-phosphonate hydrochloride (11h);

Di[4-(N-glycylamino)phenyl]-1-((S)-prolyl)-pyrrolidine-2(R,S)-phosphonate trihydrochloride (11i);

Di(4-(S)-alanylaminophenyl)-1-((S)-prolyl)pyrrolidine-2(R,S)-phosphonate trihydrochloride (11j);

Di(4-(S)-pyroglutamylaminophenyl)-1-((S)-prolyl)-pyrrolidine-2(R,S)-phosphonate hydrochloride (11k);

Di{4-[-(S)-(2-methoxycarbonyl-2-acetamido)ethyl]-phenyl}1-((S)-prolyl)pyrrolidine-2-phosphonate hydrochloride (11l);

Di(4-[(ethoxycarbonyl)methylaminocarbonyl]phenyl) 1-((S)-prolyl)pyrrolidine-2(R,S)-phosphonate (11n);

Di{4-[2-(methoxycarbonyl)ethylaminocarbonyl]phenyl}1-((S)-prolyl)-pyrrolidine-2(R,S)-phosphonate hydrochloride (11o);

Di[4-(n-propylaminocarbonyl)phenyl]1-((S)-prolyl)-pyrrolidine-2(R,S)-phosphonate hydrochloride (11p).

A compound having an inhibitory activity for DPP IV, is for example 2,2′-Biphenyl 1-((S)-prolyl)-pyrrolidine-2(R,S)-phosphonate hydrochloride (19) or a pharmaceutically acceptable salt.

Other specific compounds of group 2 (formula III) are the following:

2,2′-2-Biphenyl 1-(benzyloxycarbonyl-(S)-prolyl)-pyrrolidine-2(R,S)-phosphonate (17a);

2,2′-Biphenyl 1-(t-butyloxycarbonyl-(S)-prolyl)-pyrrolidine-2(R,S)-phosphonate (17b).

An example of a compound of group 3 (formula IV) is:

2-(2′-Hydroxyphenyl)phenyl methyl 1-(S)-prolyl)-pyrrolidine-2(R,S)-phosphonate hydrochloride (18);

The advantage of compounds of the invention over Powers et al. (supra) follows from the comparison of the properties of the leaving groups that are created from Powers' compounds and from the compounds of the invention when the inhibitors enter into a covalent binding with the serine in the active center of the peptidases. The information comes from the Merck Index, 12th edition, Merck & Co, Inc. USA (1996).

Powers' leaving groups are among others 2-methylphenol (o-cresol), 3-methylphenol (m-cresol) and 4-methylphenol (p-cresol). The compounds belong to the group of cresols, which are desinfectants. These compounds are toxic to humans. Chronic poisoning from oral or percutaneous absorption may produce digestive disturbances, nervous disorders, vertigo, skin eruptions, jaundice, oliguria, uremia. Another leaving group in Powers' compounds is 4-hydroxybenzoic acid methylester (methyl paraben, Nipagin M) which is also a preservative in foods beverages and cosmetics and of which allergic reactions are frequently observed. 1,4-Benzenediol (hydroquinone) is a photographic developer and reducer and is used as an antioxidant. At very low concentrations there is no systemic toxicity. However, ingestion of more than 1 g results in nausea, vomiting, shortness of breath, cyanosis, convulsions and collapse. It is lethal in a dose above 5 g.

The leaving groups of the compounds of the invention however, are for example 4-hydroxyacetanilide (paracetamol, compound 11e) which is used commonly as a very safe analgesic/antipyretic drug. Another leaving group is 4-hydroxyhippuric acid ethyl ester (compound 11n) of which no toxicity has been noticed, and hippuric acid is known to be a metabolite occurring in human metabolism and a normal constituent in human urine.

From the above it follows that in comparison to Powers' compounds, the compounds of the invention do not present safety problems which could obstruct their further pharmaceutical development.

The compounds of the invention can be used for the therapy of pathological states associated with excessive, impaired or unbalanced activity of said enzymes.

Starting from the available information on the correlation between particular serine protease activities and various disease states the skilled person will be able to define therapeutical utilities for the modulatory compounds of the invention. Hereinbelow examples of such disease states will be listed and support for their utility given.

Thus, the invention relates to the compounds for use as a therapeutical agent. In particular, the invention relates to the compounds for use in the treatment or prophylaxis of inflammation, vascular diseases, organ specific or systemic auto-immune diseases (e.g. Graves' disease or multiple slerosis, inflammatory bowel disease), joint diseases, muscle diseases, neurological diseases, obesity, diseases associated with benign and malign cell transformation, spreading of malignant cells, conditions of glucose-intolerance, abnormal growth or growth retardation, rejection of foreign cells or tissues after transplantation, abnormalities in blood cell development, abnormal blood clotting, pain, or diseases of the central nervous system.

Support for the pharmaceutical utility of the compounds of the invention in the above indications follows from the following publications, which demonstrate the correlation of specific serine peptidases with medical indications. Inhibition of these enzymes can thus be used as treatment or prophylaxis.

The involvement of plasma prekallikrein or kallikrein in shock is described by W. Colman in Barrett, A., et al., supra, page 147-153.

The compounds of the invention can be used for prevention and treatment of thrombosis and conjunction therapy of acute myocardial infarction by specifically inhibiting Factor X (Vlasuk, G. P. in New Therapeutic Agents in Thrombosis and Thrombolysis, Sasahara, A., Loscalzo, J., eds (1997), pages 261-283), thrombin (Shafer, J. A. in New Therapeutic Agents in Thrombosis and Thrombolysis, (supra) pages 143-157) and Factor VII (factor VII-Tissue Factor) (Shafer, J. A. in New Therapeutic Agents in Thrombosis and Thrombolysis, (supra) pages 225-260).

Various serine peptidases are known to be involved in inflammation. That this is the case is described for elastases (Bank U. et al., in Cellular Peptidases in Immune Function and Diseases, Ansorge, S. & Langner, J., Plenum Press (1997), pages 231-242, D. Farley et al. in Lauwers, A. and Scharpd, S. eds., supra), kallikrein (Chao, J. in Barrett et al., supra, pages 97-101; Naidoo, Y. & Bhoola, K. in The kinin System, Framer, S. G., Academic Press (1998), pages 187-197); and Erdos, E. G. & Skidgel, R. A. in The Kinin System, supra, pages 112-141), cathepsin G (Flad, H. D. et al., in Cellular Peptidases in Immune Function and Diseases, supra, pages 223-230), DPP IV (Tanaka, S. et al., Immunopharmacology 40 (1998), 21-26), and granzymes (Berthou, C. et al., Pathol. Biol. 46 (1998), 617-624).

DPP IV (Cheng, H. C. et al. J. Biol. Chem. 273 (1998) 24207-24215), PO (Goossens et al., Eur. J. Clin. Chem. Clin. Biochem. 34 (1996), 17-22; and Ishino, T. et al., J. Biochem. 123 (1998), 540-545) and urokinase are involved in tumorigenesis and metastasis.

Holst, J. J. & Deacon, C. have described the potential of DPP IV inhibition in Type 2 diabetes or glucose intolerance (Diabetes 47 (1998), 1663-1670).

Elastase plays a role in autoimmunity diseases such as rheumatoid arthritis (Momohara, S. et al., Clin. Rheumatol. 16 (1997), 133-140; and Shinguh, Y. et al., Eur. J. Pharmacol. 337 (1997), 63-71; Barrett, supra, Lauwers & Scharpé, supra).

Korom, S. et al., (Transplantation 63 (1997), 1495-1500 have found an involvement of DPP IV in transplant rejection. Likewise, DPP IV has been found to be involved in growth abnormalities (Bai, P. & Chang, L. L., J. Pharm. Pharmacol. 47 (1995), 698-701; and Martin, R. et al., Biochim. Biophys. Acta 1164 (1993), 252-260) and hypertension and pre-eclampsia (Neudeck, H. et al., J. Reproductive Immunology 37 (1997), 449-458). The latter two indications are also correlated with PO (Chappell, M. C. et al., Braz. J. Med. Biol. Res. 31 (1998), 1205-1212; and Umemura, K. et al., Br. J. Clin. Pharmacol. 43 (1997), 613-618).

PO is furthermore involved in muscle dystrophy (Kar, N. & Pearson, C., Clin. Chim. Acta 111 (1981), 271-273) and behavioral and neurochemical diseases (Toide, K. et al., Reviews in Neurosciences 9 (1998), 17-29).

DPP IV is a very versatile enzyme according to its localisation and different substrates and correlated with pain (Shane, R. et al., Brain Res. 815 (1999), 278-286) and obesity (Pederson, R. et al., Diabetes 47 (1998) 1253; Flint, A. et al., J. Clin. Investigation 101 (1998), 515-520) and tissue repair (Drucker, D. et al., Am. J. Physiol. 276 (1999), G79-91).

The present invention also includes derivatives which have been modified in the N-terminal amino acid side chain without abolishing the reactivity with the active site. Examples of such modifications are the incorporation of a radioactive label such as Iodine¹²⁵ into tyrosine, extension of the side chain to attach biotin or a fluorophore. To improve the half-life in the circulation the peptide bond between the two amino acids may be replaced by a non-hydrolyzable bond. The incorporation of a radioactive label is useful in diagnostic methods using the modulating compounds.

The compounds are for example labeled for use in diagnostic and research methods such as fluorescence and radio-assays, imaging, in situ histochemical and cytochemical staining etc. as will be further explained hereinbelow.

According to a further aspect thereof, the invention relates to the use of the compounds for the preparation of a therapeutical composition for modulating (inhibiting or stimulating) the activity of serine proteases. Such therapeutical composition is then specifically intended for treatment and prophylaxis of the conditions listed above.

Furthermore, the invention provides a pharmaceutical preparation comprising one or more compounds of the invention and a suitable excipient, carrier or diluent. Such pharmaceutical preparations are intended for the treatment and prophylaxis of the above conditions.

Acceptable excipients, carriers and diluents are well known and are described, for example, in Remington's Pharmaceutical Sciences, Mack Publishing Co. (A. R. Gennaro edit. 1985). Preservatives, stabilizers, dyes and flavoring agents may be provided in the pharmaceutical compositions. For example, sodium benzoate, sorbic acid and esters of p-hydroxybenzoic acid may be added as preservatives. In addition antioxidants and suspending agents may be used.

The compounds of this invention may be formulated and used as tablets, capsules or elixirs for oral administration, suppositories for rectal administration, sterile solutions or suspensions for injectable administration, aerosols, galenic preparations for topical and bucal administration and aerosols for nasal administration. If desired, absorption enhancing preparations (e.g. liposomes) or other appropriate delivery systems may be used. The amount of the active substance(s) in a dosage unit may vary between 0.001 mg and 1 g.

The compounds or pharmaceutical compositions of this invention can be used alone or in combination with one another, or in combination with other therapeutic or diagnostic agents. Such other therapeutic or diagnostic agent(s) can have a different dosage form or can be present in the same dosage form as the compounds of the invention. The dosage for the compounds of the present invention can range broadly depending upon the desired effects and the therapeutic indication.

The invention also relates to a method for in vitro inhibition or stimulation (modulation) of protease activity by means of a suitable concentration of a compound of the invention. Such method is in particular useful when a protease inactivates a peptide prior to measurement thereof in a peptide assay. The compounds of the invention can be used to inhibit the degradation of the peptide substrate by the enzyme in such assay.

The compounds of the invention are also useful in a method for the ‘ex vivo’ inhibition of protease activity, such as the treatment outside the body of cells and organs for transplantation in order to avoid rejection thereof by the recipient body.

Furthermore, the compounds of the invention can be used in a method for in vivo inhibiting or stimulating (modulating) protease activity by means of administering to a living organism a suitable amount of a compound of the invention.

Such modulation (inhibition or stimulation) can be used for pharmacotherapy of disease states related to one of the following conditions: inflammation, organ specific or systemic auto-immune diseases, non-malignant disorder of leukocytes and/or immunoglobulins, rejection of cells or tissues after transplantation, tissue destructive and bone degenerative diseases, neuroendocrine dysfunction, glucose-intolerance, obesitas, functional gastrointestinal disorder, abnormal growth or growth retardation, thrombosis and hemorrhage, vascular and cardiopulmonary diseases, neurodegenerative and affective disorders, pain, diseases associated with neoplasia.

As already indicated above, the invention also relates to the diagnostic use of the compounds. Labeled inhibitors or stimulators can be used essentially in the same type of applications as labeled monoclonal antibodies, e.g. fluorescence and radioassays, cytofluorimetry, fluorescence activated cell sorting etc. The principles of such techniques can be found in immunochemistry handbooks, for example: Coligan, J. et al., Current Protocols in Immunology, vol. 1, 2 & 3, Wiley, 1998.

The compounds of the present invention can also be used as affinity ligands for analytical and preparative purposes.

In cytochemistry and histochemistry labeled modulators can be used to directly visualize the cellular distribution of the target protease. The label can be fluorescent for fluorescence microscopy, radioactive for autoradiography, or electron dense for electron microscopy. The target structures can be whole cells, cells fixed onto slides or sections through solid tissue. A useful modification of these techniques is to use an indirect (“sandwich”) assay employing the specific high affinity interaction between biotin and avidin (Coligan et al., supra).

For imaging or therapeutic targeting of tumours expressing high amounts of the target protease, modulators labeled with a suitable isotope can be injected. Eventually, after clearing of the excess modulator from the circulation, the tumour can be visualized by radioscintigraphy. The principles of imaging are summarized by A. Bamias & A. A. Epenetos (1995), Monoclonal antibodies, production, engineering and clinical application (M. A. Ritter and M. M. Ladyman, eds.) Cambridge University Press, Cambridge, pp. 222-246).

The modulators of the invention are suitable for the diagnostic applications described above, such as imaging and histochemical staining of the respective proteases end peptidases, because they form covalent, long-lived adducts with the proteases and peptidases. Because of their small size they are expected to penetrate tissue more easily than, for example, antibodies. In the case of DPP IV it was found that the modulators only recognize DPP IV which is native and enzymatically active Formulations of the compounds to be used in diagnostic applications are also part of this invention.

Localisation of DPP IV by means of monoclonal antibody and by enzymatic activity is diagnostically useful and applicable to clinical material in the case of thyroid follicular tumours and thyroid papillary carcinoma (Kotani, T. et al., Int. J. Exp. Path. 73, 215-222 (1992); Kotani, T. et al., J. Path. 168, 41-45 (1992). In an analogous manner the compounds of the invention which form a stable adduct with DPP IV or other serine proteases may be used as a tool for diagnosing of certain disease states or monitoring the progression thereof or of treatments (Kurktschiev, D. et al., Clin. Exp. Immunol. (1999) 115, 144-146).

The invention is not limited to the compounds as claimed but also relates to pharmaceutically acceptable salts thereof.

The compounds of the invention may be pure diastereo-isomers or racemic mixtures.

Hereinbelow the invention will be described in more detail. The Examples disclose the synthesis of the modulating compounds of the invention, the inhibitory or stimulatory (modulatory) activity of the compounds of the invention on the catalytic activity of human DPP IV, PO, DPP II and elastase.

Serine protease activity can interfere with enzymatic assays for other substrates by cleaving the substrate used in the test and thereby giving either false positive (when a chromogenic substrate is cleaved) or false negative results (when a peptide substrate is degraded). The inhibitors of this invention can be used to inactivate contaminating serine proteases or peptidases before carrying on with the analysis.

This application relates to compounds that can either inhibit or stimulate the activity of serine proteases and peptidases. Because the activity of individual compounds of the group of compounds may be opposed (i.e. either inhibiting or stimulating) the general terms “modulating” and “modulation” are intended when reference is made to a group of compounds. Individual compounds will have either a inhibitory or a stimulatory activity on individual serine peptidases and proteases. A prerequisite of the invention is that the compounds that are claimed are active, i.e. have a modulatory (either inhibitory or stimulatory) activity on serine peptidases or serine proteases.

The terms “modulator”, “inhibitor”, “stimulator”, “compound”, “derivative” and “modulating compound” are used interchangeably.

The present invention will be further elucidated with reference to the following examples which are only given for illustration purposes and are in no way intended to limit the invention. The examples show how the previously reported diphenyl 1-(S)-prolyl-pyrrolidine-2-(R,S)-phosphonate (5) was used as a lead compound for the development of potent and irreversible inhibitors of dipeptidyl peptidase IV (DPP IV, EC 3.4.14.5). The synthesis of a series of diaryl 1-(S)-prolylpyrrolidine-2-(R,S)-phosphonates with different substituents on the aryl rings started from the corresponding phosphites. A good correlation was found between the electronic properties of the substituent and the inhibitory activity and stability. The most striking divergence of this correlation was the high potency combined with a high stability of the 4-acetylamino substituted derivative (11e). For this compound no cytotoxicity in human peripheral blood mononuclear cells could be observed and it also has favourable properties in vivo. Therefore di(4-acetamidophenyl) 1-(S)-prolyl-pyrrolidine-2-(R,S)-phosphonate (11e) is considered as a major improvement and will be a highly valuable DPP IV inhibitor for further studies on the biological function of the enzyme and the therapeutic value of its inhibition.

In the Examples reference is made to the following schemes and figures:

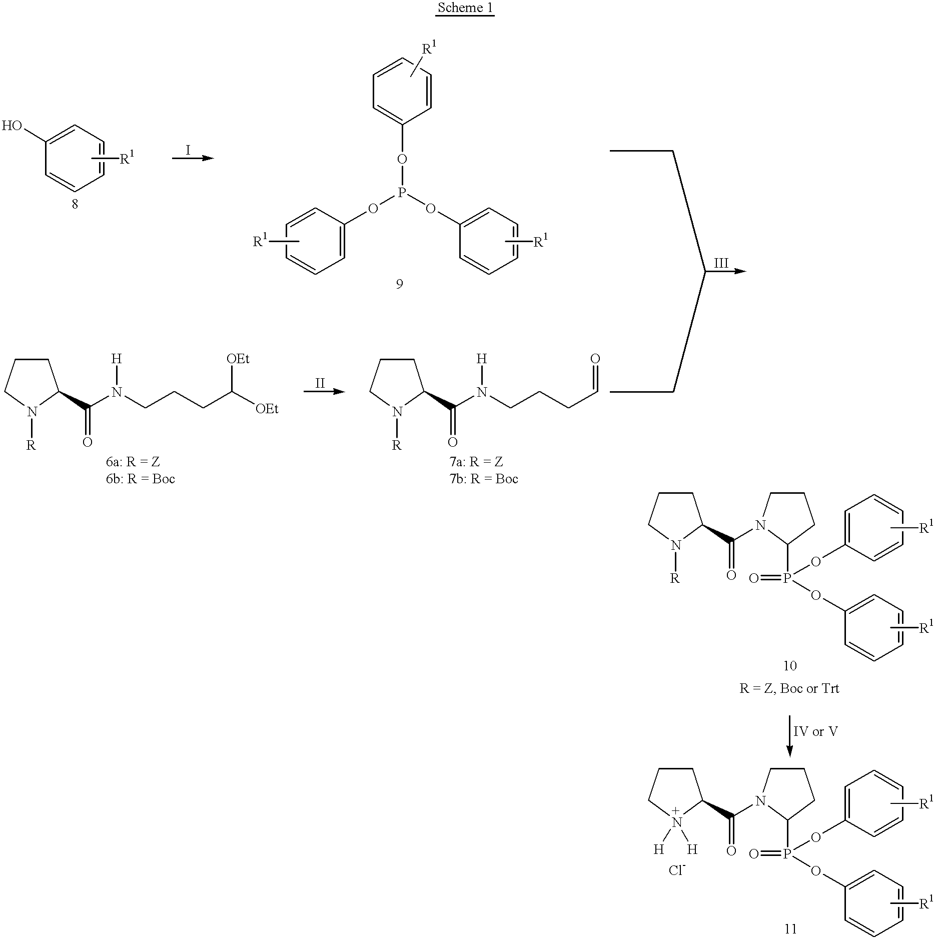

Reagents: i) PCl₃; ii) HCl; iii) HOAc, 90° C., 2 h; iv) HCl/EtOAc (1 M); v) H₂, Pd/C

For 8-11: R1=a) H; b) 4-OMe; c) 4-OAc (4-OH for 11); d) 3-NHAc; e) 4-NHAc; f) 4-NHSO₂Me; g) 3-NHCONH₂; h) 4-(N—Bz—Gly—NH); i) 4-(N—Z—Gly—NH) [4-(H—Gly—NH) for 11]; j) 4-(N—Z—(S)—Ala—NH) [4-(H—(S)—Ala—NH) for 11]; k) 4-((S)—Pyr—NH); 1) 4-[(2S)—MeO₂CCH(NHAc)CH₂]; m) 4-COOMe ; n) 4-(CONHCH₂COOEt); o) 4-[CONH(CH₂)2COOMe]; p) 4-(CONH(CH₂)2CH₃).

[chemical structure]

Reagents: i); ii) ACOH, Et₃N; iii) 7, HOAc, 90° C., 2 h; iv) H2, Pd/C; v) HCl/EtOAc (1 M).

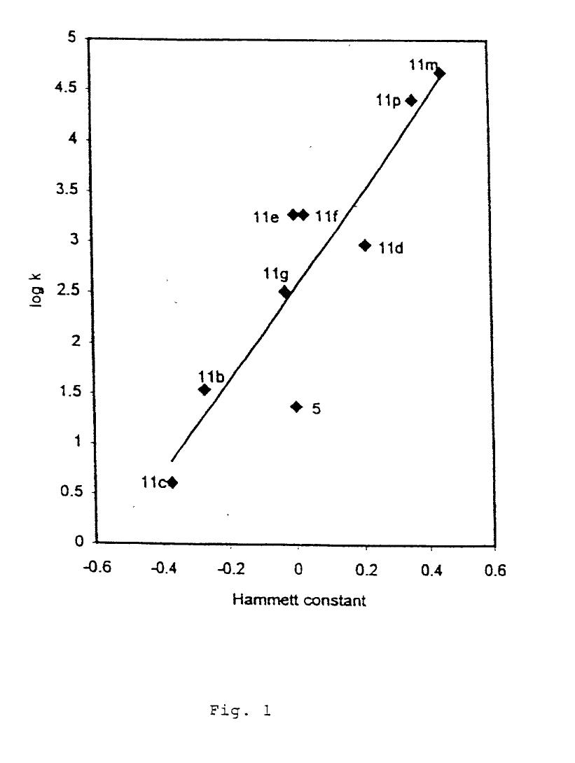

FIG. 1. Correlation between Hammett constant and log k_{calc}.

A good correlation (log k=4.6504σ+2.5472, r²=0.82) was observed between the Hammett constant and log kcalc, the most striking divergence being the difference between the unsubstituted (5) and the 4-acetylamino (11e)

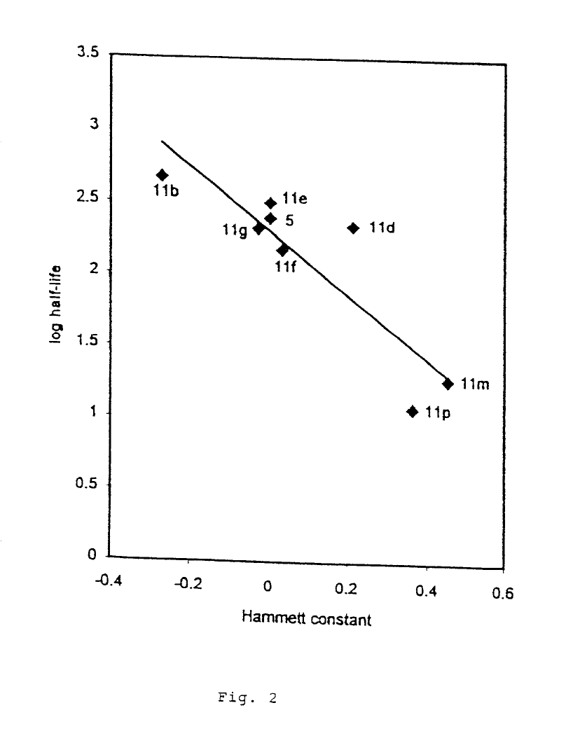

FIG. 2. Correlation between Hammett constant and log half-life.

A good correlation (log t_{½}=−2.2138σ+2.3043, r²=0.77) was observed between the Hammett constant and log t_{½}.

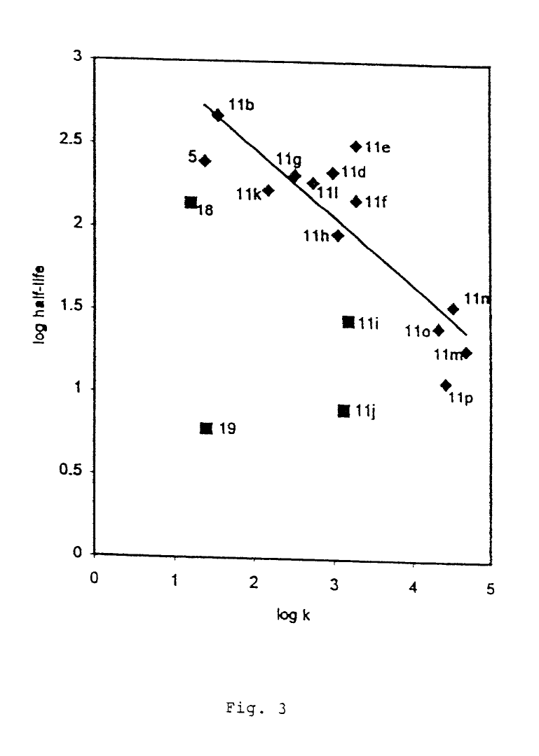

FIG. 3. Correlation between log k_{calc} and log half-life.

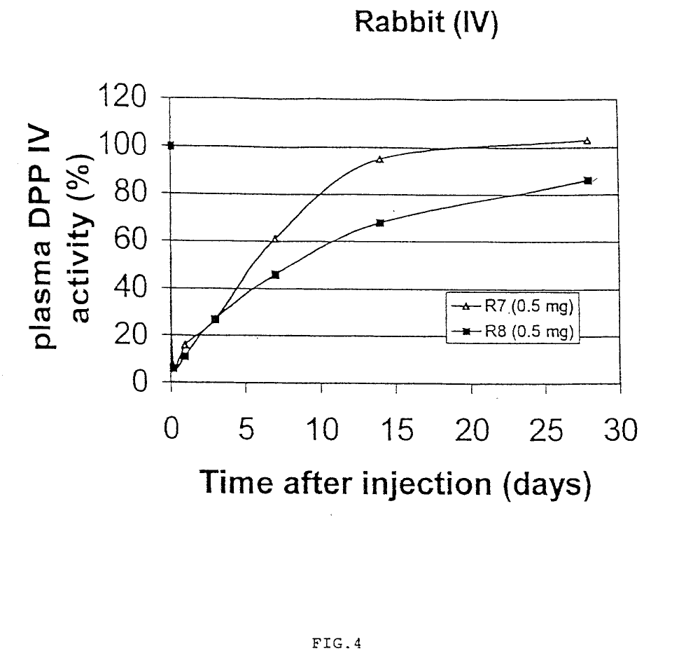

FIG. 4. Plasma DPP IV activity in rabbits treated with 11n. “R7” and “R8” are rabbits no. 7 and 8.

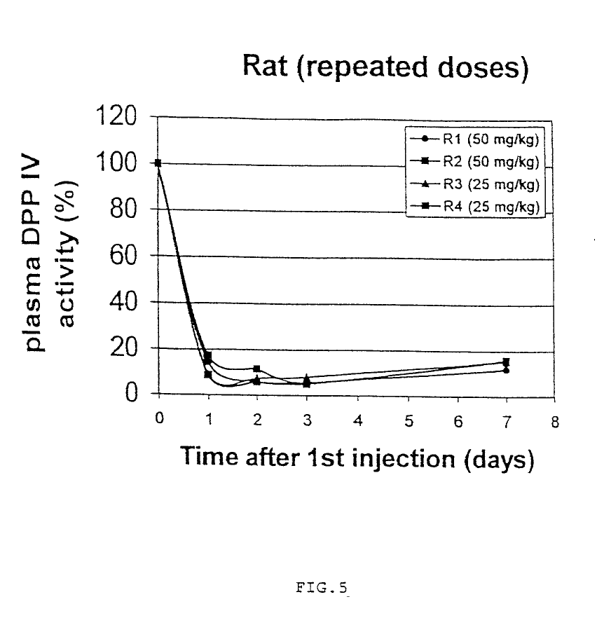

FIG. 5. Residual plasma DPP IV activity in rats treated subcutaneously on days 0 to 5 with 11e. The indications “R1”, “R2”, “R3” and “R4” identify the various rats.

A good correlation (log t_{½}=−0.4051 (log k)+3.2899, r²=0.75) was observed between log half-life and log k. Compounds 11i, 11j, 18 and 19 were left out. Compounds 11i and 11j have a considerable shorter half-life than could be expected, presumably because they suffer from another metabolism, extra to the phosphonate ester hydrolysis (see discussion). Compounds 18 and 19 cannot be compared directly to the other diaryl phosphonate esters.

### EXAMPLES

### Example 1

Synthesis of a Series of Protected and Unprotected Prolylpyrrolidine Diaryl Phosphonates

The synthesis of a series of prolylpyrrolidine diaryl phosphonates started with the coupling of 4-aminobutyraldehyde diethyl acetal to N-protected proline, activated as mixed anhydride with isobutyl chloroformate, as published previously (Belyaev, A. et al. A New Synthetic Method for Proline Diphenyl Phosphonates. Tetrahedron Lett. 1995, 36, 3755-3758). The acetal 6 was hydrolysed with HCl, and the crude aldehyde 7 was treated with the corresponding triaryl phosphite 9 in acetic acid to give diastereoisomeric mixtures of the protected prolylpyrrolidine diaryl phosphonate 10 (Belyaev et al., (1995), supra) (Scheme 1). Deprotection using standard methods afforded the final compounds 11.

For the preparation of substituted phenyl phosphonates, it was necessary to prepare some commercially unavailable phenols. The 4-methylsulfonylaminophenol 8f was prepared from the corresponding sulfonylchloride and 4-aminophenol. Likewise, the 4-acylaminophenols 8h-k were prepared by condensation of the corresponding carboxylic acid with 4-aminophenol using the mixed anhydride method. N-acetyl-L-tyrosine methyl ester (8l) was prepared as described (Jackson, E. L. O-p-Toluenesulfonyl-L-tyrosine, its N-acetyl and N-benzoyl Derivatives. J. Am. Chem. Soc. 1952, 74, 837-838). The glycine derivative 8n was obtained after condensation of glycine ethyl ester with 4-hydroxybenzoic acid using diphenylphosphoryl azide (DPPA). The synthesis of the 4-hydroxybenzoic acid amides 8o and 8p was accomplished by coupling of 4-acetoxybenzoic acid with the corresponding amine using the mixed anhydride method followed by mild alkaline hydrolysis of the phenyl esters 12 and 13 (Büchi, G.; Weinreb, S. M. Total Syntheses of Aflatoxins M1 and G1 and an Improved Synthesis of Aflatoxin Bi. J. Am. Chem. Soc. 1971, 93, 746-752). The triaryl phosphites 9 were then synthesised from the corresponding substituted phenols 8 and phosphorous trichloride.

Cyclic N-protected 2,2,′-biphenyl derivatives 17a and 17b were prepared by reacting 2,2′-biphenylacetyl phosphite (16) with aldehyde 7a or 7b in acetic acid (Scheme 2). Attempted removal of benzyloxycarbonyl (Z) protection from phosphonate 17a by hydrogenolysis with a Pd/C catalyst in methanol resulted in the opening of the dioxaphosphepan ring to give the mixed methyl aryl ester 18. Deprotection of the Boc-derivative 17b with HCl/EtOAc yielded free 2,2′-biphenyl phosphonate 19.

Generally, with this method we obtained a mixture of diastereoisomers of the phosphonates 10. To obtain the pure diastereoisomers of 5, it was necessary to introduce a trityl protection after removal of the Z-protection, which resulted in easily separable isomers (10a(S,R) and 10a(S,S)). We suppose that the most active isomer has the R configuration at the carbon atom next to phosphorus, and this was confirmed by comparison of the relative mobility on TLC, optical activity, ¹H-NMR spectrum and biological activity with that of (S)—Ile—(R)—ProP(OPh)2. The configuration of this reference compound was unambiguously determined with X-ray crystallography (Belyaev et al., (1995), supra).

Experimental

General. 4-methoxyphenol (8b), 1-(3-hydroxyphenyl)urea (8g), methyl 4-hydroxybenzoate (8m), triphenyl phosphite (9a), 2,2′-biphenol (14), L-proline, L-tyrosine, 4-aminophenol, glycine, L-pyroglutamic acid, 4-hydroxybenzoic acid, glycine ethyl ester hydrochloride, β-alanine and all common chemicals were purchased from Acros Chimica N. V., Belgium. 3-Acetamidophenol (8d), 4-aminobutyraldehyde diethyl acetal, PCl₃, diphenylphosphoryl azide (DPPA) and 4-acetoxybenzoic acid were obtained from Sigma-Aldrich Chemie BV, Belgium. 4-Acetamidophenol (paracetamol) (8e) was obtained from Sterling Organics Ltd., England. Purity of all new synthesised compounds were checked by TLC, ¹H-NMR, ¹³C-NMR or MS. The final products were checked by TLC, ¹H-NMR, ¹³C-NHR, FAB-MS and(or) elemental analysis. Thin-layer chromatography was performed with POLYGRAM(r) SIL G/UV254 plates precoated with silica gel (Machinery-Nagel GmbH, Germany) using EtOAc-petroleum ether, EtOAc-MeOH or n-BuOH-AcOH-H₂O (4:1:1) mixtures as eluent. Silica gel H. 5-40 μm (Fluka, Switzerland) was used for preparative vacuum column chromatography. The NMR spectra were recorded on a Varian EM360L or a Brucker Avance DRX 400 spectrometer. Mass-spectra were recorded on a VG 70-SEQ spectrometer. Optical rotation was measured on a Perkin-Elmer 241 polarimeter. Melting points were determined on a Digital Melting Points Apparatus (Electrothermal) and are uncorrected. Z- and Boc-protected amino acids were prepared according to standard procedures (Bodanszky, M. & Bodanszky, A. In The Practice of Peptide Synthesis, 2nd ed.; Springer-Verlag: Berlin Heidelberg, 1994; pp 11-18 and 28-29) using Z-Cl and (Boc)₂O, respectively. β-Alanine methyl ester was synthesised by treatment of the amino acid in methanolic HCl similarly to described procedure (Bodanszky & Bodanszky (1994), supra). The preparation of 4-(Z-(S)-prolyl)aminobutyraldehyde diethyl acetal (6a) was described earlier (Belyaev et al., (1995), supra). 2-Chloro-(dibenzo[d;f]-1,3,2-dioxaphosphepan) (15) was prepared as described (Veriznikov, L. V. & Kirpichnikov, P. A. Synthesis of the o,o′-Biphenylphosphinic Acid Esters. Zh. Obshch. Khim. 1967, 37, 1355-1358).

4-(Boc-(S)-prolyl)aminobutyraldehyde diethyl acetal (6b).

To a solution of Boc-(S)-Proline (10 mmol, 2.14 g) in dry CHCl₃ (20 mL), Et₃N (10 mmol, 1.4 mL) was added with stirring at −10° C. followed by isobutyl chloroformate (10 mmol, 1.31 mL). After 20 min, 4-aminobutyraldehyde diethyl acetal (10 mmol, 1.73 mL) was added and the mixture was stirred overnight (−10° C. to room temperature). Chloroform was removed, the residue was distributed between EtOAc (100 mL) and water (100 mL), the organic layer was dried (MgSO₄) and purified by column chromatography. Yield 2.96 g (83%): oil; ¹H-NMR (CDCl₃) δ (ppm) 1.0-2.4 (m, 23H, 3- and 4-CH₂ (Pro), CH₂CH₂, C(CH₃)₃, CH₃), 2.9-3.9 (m, 9H, 5-CH₂, NCH₂, OCH₂ and (EtO)2CH), 4.25 (m, ¹H, 2-CH), 4.5 (t, ¹H, NH).

4-Methylsulfonylaminophenol (8f).

To a suspension of 4-aminophenol (200 mmol, 21.8 g) in MeOH (250 mL), CH₃SO₂Cl (100 mmol, 7.75 mL) was added at 10-15° C. with stirring. The resulting solution was stirred for 1 h at room temperature and evaporated. The residue was suspended in 1N HCl (250 mL), the solid was filtered, washed with water and dried in vacuum over NaOH. Yield 13.6 g (81%): mp 165-166° C.; ¹H-NMR (DMSO) δ (ppm) 2.85 (s, 3H, CH₃S), 6.70 (d, 2Hₐᵣₒₘ), 7.10 (d, 2Hₐᵣₒₘ), 8.70 (s, ¹H, OH), 8.85 (s, ¹H, NH).

Synthesis of 4-acylaminophenols 8h-k

General procedure.

To a solution of carboxylic acid (100 mmol) in DMF (100 mL), Et₃N (100 mmol, 14 mL) was added at −10° C., followed by isobutyl chloroformate (100 mmol, 13 mL). After 0.5 h at this temperature, 4-aminophenol (115 mmol, 12.5 g) was added and the mixture was stirred overnight (−10° C. to room temperature). After dilution with 1N HCl (500 mL), the precipitate was collected by filtration, and washed on the filter with water, NaHCO₃ solution, water and, finally, ether. The product was dried and, if necessary, recrystallized from an appropriate solvent.

N2-Benzoyl-N-(4-hydroxyphenyl)glycinamide (8h).

Recrystallized from EtOH-acetone. Yield 35%: mp 244-245° C.; ¹H-NMR (DMSO) δ (ppm) 4.05 (d, 2H, CH₂CO), 6.5-8.0 (m, 9Hₐᵣₒₘ), 8.7 (m, ¹H, NH), 9.15 (s, ¹H, OH), 9.75 (s, ¹H, NH).

N2-Benzyloxycarbonyl-N-(4-hydroxyphenyl)glycinamide (8i).

Yield 44%: mp 184-185° C.; ¹H-NMR (DMSO) δ (ppm) 3.80 (d, 2H, CH₂CO), 6.5-7.5 (m, 10H, 9Hₐᵣₒₘ and NH), 8.85 (s, ¹H, OH), 9.40 (s, ¹H, NH).

N2-Benzyloxycarbonyl-N-(4-hydroxyphenyl)-(S)-alaninamide (8j)

Yield 73%: mp 97-100° C.; ¹H-NMR (DMSO) δ (ppm) 1.36 (d, 3H, CH₃), 4.29 (m, ¹H, CH of L-Ala), 5.00 (s, 2H, CH₂), 6.00 (m, ¹H, NH), 6.35-7.38 (m, 9H, Hₐᵣₒₘ) 8.69 (br s, ¹H, OH), 9.24 (br s, ¹H, NHCO)

N-(4-Hydroxyphenyl)-(S)-pyroglutamylamide (8k).

Yield 64%: mp 303-305° C. dec; ¹H-NMR (DMSO) δ (ppm) 1.7-2.7 (m, 4H, CH₂CH₂), 4.2 (m, ¹H, CHCO), 6.65 (d, 2Hₐᵣₒₘ), 7.40 (d, 2Hₐᵣₒₘ), 7.8 (s, ¹H, NH), 9.1 (br s, ¹H, OH), 9.7 (s, ¹H, NH).

N-(4-Hydroxybenzoyl)glycine ethyl ester (8n).

To a solution of 4-hydroxybenzoic acid (100 mmol, 13.8 g) in DMF (70 mL), H-Gly—OEt.HCl (100 mmol, 14 g) was added followed by DPPA (100 mmol, 21.6 mL) and Et₃N (200 mmol, 28 mL) with stirring at 0° C. The mixture was stirred overnight (0° C. to room temperature), Et₃N.HCl was removed by filtration and the filtrate was evaporated to half of the initial volume. The residue was mixed with 5% NaHCO₃ (200 mL) and extracted with EtOAc (500 mL). The organic layer was washed with brine, dried (MgSO₄) and evaporated until crystallisation. The mixture was diluted with ether (100 mL), and the crystals were collected. Yield 9 g (40%): mp 205-209° C.; ¹H-NMR (DMSO) δ (ppm) 1.25 (t, 3H, CH₃), 4.05 (m, 4H, CO₂CH₂ and COCH₂N), 6.8 (d, 2Hₐᵣₒₘ), 7.75 (d, 2Hₐᵣₒₘ), 8.35 (tr, ¹H, NH), 9.7 (s, ¹H, OH).

Synthesis of 4-hydroxybenzoic acid amides 8o and 8p

General procedure.

To a solution of 4-acetoxybenzoic acid (100 mmol, 18 g) in THF (250 mL), Et₃N (100 mmol, 14 mL) was added followed by isobutyl chloroformate (100 mmol, 13 mL) with stirring at −10° C. After 15 min the corresponding amine (free base or HCl salt) was added followed by Et₃N (100 mmol, 14 mL) in case of a hydrochloride. The mixture was stirred overnight (−10° C. to room temperature), the solid was removed by filtration, the filtrate was evaporated and the residue crystallised from an ether-hexane mixture. 4-Acetoxybenzoic acid amide 12, 13 thus obtained (70 mmol) was dissolved in MeOH (240 mL),

H₂O (90 mL) was added followed by saturated NaHCO₃ solution (120 mL). An excess of NaHCO₃ precipitated. The heterogeneous mixture was stirred for 4 h at room temperature, methanol was removed in vacuum, the residue was extracted with EtOAc (250 mL), the organic layer was washed with 1N HCl, brine, then dried (MgSO₄) and evaporated. The residue crystallised from ether or was purified on a silica gel column.

N-(4-Acetoxybenzoyl)-B-alanine methyl ester (12).

Yield 69%: mp 84-88° C.; ¹H-NMR (CDCl₃) δ (ppm) 2.35 (s, 3H, COCH₃), 2.6 (t, 2H, CO₂CH₂), 3.7 (m, 5H, NCH₂ and CO₂CH₃), 7.15 (m, 3H, 2Hₐᵣₒₘ and NH), 7.75 (d, 2Hₐᵣₒₘ).

N-n-Propyl-4-acetoxybenzoylamide (13).

Yield 73%: mp 94-98° C.; ¹H-NMR (CDCl₃) δ (ppm) 0.9 (t, 3H, CH₃), 1.6 (m, 2H, CH₂), 2.25 (s, 3H, COCH₃) 3.4 (m, 2H, NCH₂), 7.1 (m, 3H, 2Hₐᵣₒₘ and NH), 7.8 (d, 2Hₐᵣₒₘ).

N-(4-Hydroxybenzoyl)-β-alanine methyl ester (8o).

Yield 65%: mp 124-126° C.; ¹H-NMR (DMSO) δ (ppm) 2.55 (t, 2H, CO₂CH₂), 3.7 (m, 5H, NCH₂ and CO₂CH₃), 6.9 (m, 3H, 2Hₐᵣₒₘ and NH), 7.55 (d, 2Hₐᵣₒₘ), 8.7 (br s, ¹H, OH).

N-n-Propyl-4-hydroxybenzoylamide (8p).

Yield 78%: oil; ¹H-NMR (DMSO) δ (ppm) 1.1 (t, 3H, CH₃), 1.55 (m, 2H, CH₂), 3.3 (m, 2H, NCH), 6.9 (m, 3H, 2Hₐᵣₒₘ and NH), 7.65 (d, 2 Hₐᵣₒₘ), 8.5 (br s, ¹H, OH); MS (FAB+) m/z 180 (M+H)⁺.

Synthesis of triaryl phosphites 9

General procedure.

To a stirred solution of the corresponding phenol (90 mmol) and Et₃N (90 mmol, 12.6 mL) in dry DMF (30 mL), a solution of PCl₃ (30 mmol, 2.62 mL) in dry CHCl₃ (10 mL) was added dropwise at 0° C. The mixture was stirred overnight (0° C. to room temperature), diluted with CHCl₃ (500 mL) and the resulting solution was washed with water (2×500 mL). The organic layer was dried (MgSO₄), evaporated and the residue, eventually together with the product insoluble in both layers, was purified by column chromatography with an EtOAc—methanol or an EtOAc—petroleum ether mixture as eluent.

Tris(3-acetamidophenyl) phoshite (9d).

Yield: 42%: solid foam; ¹H-NMR (DMSO) δ (ppm) 2.1 (s, 9H, COCH₃), 6.7-7.7 (m, 12Hₐᵣₒₘ), 9.7 (s, 3H, NH).

Tris(4-acetamidophenyl) phosphite (9e).

Yield 37%: solid foam; ¹H-NMR (DMSO) δ (ppm) 2.1 (s, 9H, COCH₃), 6.9-7.7 (dd, 12Hₐᵣₒₘ), 9.2 (s, 3H, NH).

Tris(4-methylsulfonylaminophenyl) phosphite (9f).

Yield 60%: solid foam; ¹H-NMR (DMSO) δ (ppm) 2.95 (s, 9H, SO₂CH₃), 7.2 (m, 12Hₐᵣₒₘ), 9.15 (s, 3H, NH).

Tris(3-ureylphenyl) Phosphite (9g).

Yield 30%: solid foam; ¹H-NMR (DMSO) δ (ppm) 5.85 (m, 6H, CONH₂), 6.5-7.5 (m, 12Hₐᵣₒₘ)l 8.65 (s, 3H, NH).

Tris [4-(N-benzoylglycylamino)phenyl]phoshite (9h).

Yield 90%: mp 210° C. (dec.); ¹H-NMR (DMSO) δ (ppm) 4.1 (d, 6H, COCH₂N), 6.9-8.0 (m, 27Hₐᵣₒₘ), 8.5 (m, 3H, NH), 9.9 (s, 3H, NH).

Tris[4-(N-benzyloxycarbonylglycylamino)phenyl]phosphite (9i).

Yield 76%: mp 173° C. (dec.); ¹H-NMR (DMSO) δ (ppm) 3.85 (d, 6H, COCH₂N), 5.05 (s, 6H, CH₂Ph), 6.5-8.0 (m, 30H, NH and Hₐᵣₒₘ), 98 (br s, 3H, NH).

Tris[4-(N-benzyloxycarbonyl-(S)-alanylamino)phenyl]phosphite (9j).

Yield: 85%: solid foam; ¹H-NMR (DMSO) δ (ppm) 1.42 (d, 9H, CH₃), 4.29 (m, 3H, CH), 5.01(s, 6H, CH₂Ph), 6.45-7.15 (d, 3H, NH), 7.25 (m, 27H, Hₐᵣₒₘ), 9.76 (br s, 3H, NH).

Tris[4-((S)-pyroglutamylamino)phenyl]phosphite (9k).

Yield 31%: solid foam; ¹H-NMR (DMSO) δ (ppm) 1.8-2.8 (m, 12H, CH₂CH₂), 4.25 (m, 3H, COCHN), 7.1 (d, 6Hₐᵣₒₘ), 7.65 (d, 6Hₐᵣₒₘ), 8.25 (s, 3H, NH), 10.3 (s, 3H, NH).

Tris{4-[-(S)-(2-methoxycarbonyl-2-acetamido)ethyl]phenyl}phosphite (9l).

Yield 73%: solid foam; ¹H-NMR (DMSO) δ (ppm) 2.0 (s, 9H, COCH₃), 3.1 (d, 6H, ArCH₂), 3.7 (s, 9H, CO₂CH₃), 4.8 (m, 3H, COCHN), 6.6 (d, 3H, NH), 7.1 (br s, 12Hₐᵣₒₘ).

Tris(4-methoxycarbonylphenyl) phosphite (9m).

Yield: 57%: oil; ¹H-NMR (CDCl₃) δ (ppm) 3.95 (s, 9H, CO₂CH₃), 6.65-8.05 (m, 12Hₐᵣₒₘ).

Tris{4-[(ethoxycarbonyl)methylaminocarbonyl]phenyl}phosphite (9n). Yield 70%: solid foam; ¹H-NMR (CDCl₃) δ (ppm) 1.3 (t, 9H, CH₃), 3.9-4.3 (m, 12H, COCH₂N and OCH₂), 6.7-8.0 (m, 15H, NH and Hₐᵣₒₘ).

Tris{4-[2-(methoxycarbonyl)ethylaminocarbonyl]phenyl}phosphite (9o).

Yield 71%: solid foam; ¹H-NMR (CDCl₃) δ (ppm) 2.65 (t, 6H, CO₂CH₂), 3.7 (m, 15H, NCH₂ and CO₂CH₃), 7.1 (d, 6Hₐᵣₒₘ) 7.9 (m, 9H, Hₐᵣₒₘ and NH).

Tris[4-(n-propylaminocarbonyl)phenyl]phosphite (9p).

Yield 54%: mp 207° C.; ¹H-NMR (DMSO) δ (ppm) 1.1 (t, 9H, CH₃), 1.6 (m, 6H, CH₂), 3.25 (m, 6H, CH₂N), 7.15 (d, 6Hₐᵣₒₘ), 8.1 (m, 9H, NH and Hₐᵣₒₘ).

Synthesis of N-Boc- or N-Z-Protected diaryl 1-((S)-prolyl)pyrrolidine-2(R,S)-phosphonates 10

General procedure.

Diethyl acetal 6a or 6b (10 mmol) was dissolved in a mixture of THF (60 mL) and 0.5N HCl (30 mL, 15 mmol) with stirring at room temperature. After 2 h ether (90 mL) was added with stirring and the mixture was neutralised with an excess of solid NaHCO₃ (ca. 5 g). The organic layer was separated, dried (MgSO₄) and evaporated. The residue (crude aldehyde 7a or 7b) was dissolved in acetic acid (30 mL) together with the corresponding triaryl phosphite (9, 10 mmol) and the resulting solution was stirred at 85-90° C. for 1.5-2 h. After cooling to room temperature, acetic acid was evaporated, the residue was dissolved in chloroform (250 mL), washed with water (200 mL), saturated NaHCO₃ solution (100 mL) and water (100 mL). The organic layer was dried (MgSO₄), evaporated and the residue was purified by column chromatography with an ethyl acetate-petroleum ether or ethyl acetate-methanol mixture as eluent.

Di(3-acetamidophenyl) 1-(benzyloxycarbonyl-(S)-prolyl)-pyrrolidine-2(R,S)-phosphonate (10d).

Yield 31%: solid foam: ¹H-NMR (CDCl₃) δ (ppm) 1.0-2.7 (m, 8H, 3-CH₂ and 4-CH₂), 2.1 (s, 6H, CH₃CO), 4.0-3.2 (m, 4H, 5-CH₂), 4.3-5.1 (m, 2H, 2-CH), 5.1 (br s, 2H, CH₂Ph), 6.6-7.7 (m, 13Hₐᵣₒₘ) 9.1 (s, 2H, NH); MS (FAB+) m/z 649 (M+H)⁺.

Di(4-acetamidophenyl) 1-(benzyloxycarbonyl-(S)-prolyl)-pyrrolidine-2(R,S)-phosphonate (10e).

Yield 60%: solid foam; ¹H-NMR (CDCl₃) δ (ppm) 1.6-2.7 (m, 8H, 3-CH₂ and 4-CH₂), 2.1 (s, 6H, CH₃CO), 3.3-3.8 (m, 4H, 5-CH₂), 4.3-5.1 (m, 2H, 2-CH), 5.1 (br s, 2H, CH₂Ph), 6.8-7.5 (m, 13Hₐᵣₒₘ), 8.8 (s, 2H, NH); MS (FAB+) m/z 649 (M+H)⁺.

Di(4-methylsulfonylaminophenyl) 1-(benzyloxycarbonyl-(S)-prolyl)pyrrolidine-2(R,S)-phosphonate (10f).

Yield 66%: solid foam; ¹H-NMR (CDCl₃) δ (ppm) 1.2-2.7 (m, 8H, 3-CH₂ and 4-CH₂), 2.8 (s, 6H, CH₃SO₂), 3.3-3.8 (m, 4H, 5-CH₂), 4.3-5.2 (m, 4H, 2-CH and CH₂Ph), 6.8-7.4 (m, 13Hₐᵣₒₘ), 8.0 (s, 2H, NH); MS (FAB+) m/z 721 (M+H)⁺.

Di(3-ureylphenyl) 1-(benzyloxycarbonyl-(S)-prolyl)-pyrrolidine-2(R,S)-phosphonate (10g).

Yield 8%: solid foam; ¹H-NMR (CDCl₃) δ (ppm) 1.3-2.4 (m, 8H, 3-CH₂ and 4-CH₂), 2.8-3.8 (m, 4H, 5-CH₂), 4.3-5.2 (m, 4H, 2-CH and CH₂Ph), 5.6 (m, 4H, NH₂), 6.8-7.5 (m, 13Hₐᵣₒₘ), 8.65 (br s, 2H, NH); MS (FAB+) m/z 651 (M+H)⁺.

Di[4-(N-benzoylglycylamino)phenyl]-1-(benzyloxycarbonyl-(S)-prolyl)pyrrolidine-2(R,S)-phosphonate (10h).

Yield 47%: solid foam; ¹H-NMR (CDCl₃) δ (ppm) 1.2-2.7 (m, 8H, 3-CH₂ and 4-CH₂), 3.2-3.8 (m, 4H, 5-CH₂), 4.0 (m, 4H, NCH₂CO), 4.4-5.0 (m, 2H, 2-CH), 5.1 (br s, 2H, CH₂Ph), 6.6-8.3 (m, 25H, 23Hₐᵣₒₘ and NH), 9.5 (br s, 2H, NH); MS (FAB+) m/z 887 (M+H)⁺.

Di[4-(N-benzyloxycarbonylglycylamino)phenyl]-1-(benzyloxy carbonyl-(S)-prolyl)pyrrolidine-2(R,S)-phosphonate (10i).

Yield 19%: solid foam; ¹H-NMR (CDCl₃) δ (ppm) 1.2-2.6 (m, 8H, 3-CH₂ and 4-CH₂), 3.2-4.1 (m, 8H, 5-CH₂ and COCH₂N), 4.3-5.2 (m, 8H, 2-CH and CH₂Ph), 6.1 (m, 2H, NH), 6.7-7.6 (m, 23Hₐᵣₒₘ), 8.95 (br s, 2H, NH); MS (FAB+) m/z 947 (M+H)⁺.

Di[4-(N-benzyloxycarbonyl-(S)-alanylamino)phenyl]-1-(benzyloxycarbonyl-(S)-prolyl)pyrrolidine-2(R,S)-phosphonate (10j).

Yield 40%. mp: 200-203° C.; ¹H-NMR (CDCl₃) δ (ppm) 1.35 (d, 6H, CH₃), 1.65-2.35 (m, 8H, 3-CH₂ and 4-CH₂), 2.95-3.95 (m, 4H, 5-CH₂), 4.0-4.65 (m, 4H, 2-CH, CH), 5.00 (s, 6H, CH₂Ph), 6.20 (br, 2H, NH), 7.04 (s, 23H, Hₐᵣₒₘ), 9.25 (br, 2H, NH); MS (FAB+) m/z 975 (M+H)⁺.

Di[4-((S)-pyroglutamylamino)phenyl]-1-(benzyloxycarbonyl-(S)-prolyl)pyrrolidine-2(R,S)-phosphonate (10k).

Yield 25%: mp 170° C. (dec.); ¹H-NMR (CDCl₃) δ (ppm) 1.2-2.9 (m, 16H, 3-CH₂ and 4-CH₂), 3.55 (m, 4H, 5-CH₂), 4.1 (m, 2H, 5-CH Pyr), 4.3-5.2 (m, 4H, 2-CH and CH₂Ph), 6.7-8.1 (m, 15H, NH and 13Hₐᵣₒₘ), 9.55 (br s, 2H, NH); MS (FAB+) m/z 787 (M+H)⁺.

Di{4-[-(S)-(2-methoxycarbonyl-2-acetamido)ethyl]phenyl}1-(benzyloxycarbonyl-(S)-prolyl)pyrrolidine-2(R,S)-phosphonate (10l).

Yield 57%: solid foam; ¹H-NMR (CDCl₃) δ (ppm) 1.3-2.7 (m, 8H, 3-CH₂ and 4-CH₂), 1.9 (s, 6H, COCH₃), 3.1 (d, 4H, CH₂Ar), 3.7 (s, 6H, COOCH₃), 3.2-3.9 (m, 4H, 5-CH₂), 4.5-5.0 (m, 4H, 2-CH), 5.1 (br s, 2H, CH₂Ph), 6.1 (d, 2H, NH), 7.0 (s, 8Hₐᵣₒₘ), 7.3 (br s, 5H, C₆H5); MS (FAB+) m/z 821 (M+H)⁺.

Di{4-[(ethoxycarbonyl)methylaminocarbonyl]phenyl}1-(benzyloxycarbonyl-(S)-prolyl)-pyrrolidine-2(R,S)-phosphonate (10n).

Yield 32%: solid foam; ¹H-NMR (CDCl₃) δ (ppm) 1.2-2.7 (m, 8H, 3-CH₂ and 4-CH₂), 1.3 (t, 6H, CH₃), 3.2-3.9 (m, 4H, 5-CH₂), 4.0 (m, 4H, NCH₂CO), 4.2 (q, 4H, OCH₂), 4.3-5.1 (m, 2H, 2-CH), 5.1 (br s, 2H, CH₂Ph), 6.9-7.8 (m, 15H, 13Hₐᵣₒₘ and NH); MS (FAB+) m/z 793 (M+H)⁺.

Di{4-[2-(methoxycarbonyl)ethylaminocarbonyl]phenyl}1-(benzyloxycarbonyl-(S)-prolyl)-pyrrolidine-2(R,S)-phosphonate (10o).

Yield 22%: solid foam; ¹H-NMR (CDCl₃) δ (ppm) 1.2-2.8 (m, 8H, 3-CH₂ and 4-CH₂), 2.65 (t, 4H, CH₂CO₂), 3.2-3.8 (m, 8H, 5-CH₂ and CH₂N), 3.75 (s, 6H, CO₂CH₃), 4.3-5.2 (m, 4H, 2-CH and CH₂Ph), 6.7-7.9 (m, 15H, 13Hₐᵣₒₘ and NH); MS (FAB+) m/z 793 (M+H)⁺.

Di[4-(n-propylaminocarbonyl)phenyl]1-(benzyloxycarbonyl-(S)-prolyl)-pyrrolidine-2(R,S)-phosphonate (10p).

Yield 4.3%: solid foam; ¹H-NMR (CDCl₃) δ (ppm) 0.85 (t, 6H, CH₃), 1.3-2.8 (m, 12H, 3-CH₂, 4-CH₂ and CH₂), 3.1-3.9 (m, 8H, 5-CH₂ and CH₂N), 4.5 (m, ¹H, 2-CH Pro), 4.7-5.2 (m, 3H, CH₂Ph and 2-CH ProP), 6.9-8.1 (m, 15H, 13Hₐᵣₒₘ and NH); MS (FAB+) m/z 705 (M+H)⁺.

Diaryl 1-((S)-prolyl)pyrrolidine-2-phosphonate hydrochlorides (11).

Procedure A.

The diaryl 1-(t-butyloxycarbonyl-(S)-prolyl)-pyrrolidine-2-phosphonate (10) or diaryl 1-(trityl-(S)-prolyl)pyrrolidine-2-phosphonate (10, 5 mmol) was dissolved in a 1M HCl solution in EtOAc (25 mL) and the solution was stirred for 2 h at room temperature. Dry ether (30 mL) was added and the mixture was left in the fridge overnight. If the product crystallised, the crystals were collected by filtration, otherwise the oil was triturated in dry ether. The material obtained was dried in vacuum over NaOH pellets.

Procedure B.

The diaryl 1-((S)-benzyloxycarbonylprolyl)-pyrrolidine-2-phosphonate (10, 2 mmol) was hydrogenated over Pd/C in methanol (50 mL) for 5-6 h. The catalyst was removed by filtration through celite, the filtrate was acidified with 1M HCl/EtOAc (2.2 mL), evaporated and the residue was precipitated with dry ether from methanol (5 mL). The resulting product was dried in vacuum over NaOH pellets.

Diphenyl 1-((S)-prolyl)pyrrolidine-2(R)-phosphonate hydrochloride (11a(S,R)).

A. Yield 81%: mp 175-177° C.; ¹H-NMR (CDCl₃) δ (ppm) 1.6-2.8 (m, 8H, 3-CH₂ and 4-CH₂), 3.3-3.9 (m, 4H, 5-CH₂), 4.8 (m, ¹H, 2-CH), 5.0 (m, ¹H, 2-CH), 7.2 (m, 5Hₐᵣₒₘ), 7.3 (m, 5Hₐᵣₒₘ), 8.1 (br s, ¹H, N+H), 10.2 (br s, 1H, N+H); ¹³C-NMR (CDCl₃) δ (ppm) 24.1 (4-CH₂), 24.6 (4-CH₂) 26.1 (3-CH₂), 28.8 (3-CH₂), 46.2 (5-CH₂), 47.1 (5-CH₂), 54.5 (d, 2-CH-P, 1J (C-P)=162 Hz), 59.1 (2-CH), 120.3, 125.2, 129.7, 150.3 (Cₐᵣₒₘ), 168.2 (CO); [α]D²⁰=−100.4° (C₁, CHCl₃); Anal. (C₂₁H₂₅N₂O₄P 1.25 HCl) C, H, N.

Diphenyl 1-((S)-prolyl)pyrrolidine-2(S)-phosphonate hydrochloride (11a(S,S)).

A. Yield 88%: solid foam; ¹H-NMR (CDCl₃) δ (ppm) 1.7-2.6 (m, 8H, 3-CH₂ and 4-CH₂), 3.2-3.9 (m, 4H, 5-CH₂), 4.75 (m, ¹H, 2-CH), 4.95 (m, 0.7H, 2-CH), 5.4 (m, 0.3H, 2-CH), 6.9-7.4 (m, 10Hₐᵣₒₘ), 8.6 (br s, ¹H, N+H), 10.3 (br s, 0.3H, N+H), 10.6 (br s, 0.7H, N+H); ¹³C-NMR (CDCl₃) δ (ppm) 22.1, 24.0 (4-CH₂), 23.5, 23.9 (4-CH₂), 26.4, 27.7 (3-CH₂), 28.5, 29.1 (3-CH₂), 45.6, 46.2 (5-CH₂), 46.6, 47.1 (5-CH₂), 54.4, 55.3 (d, 2-CH-P, 1J (C-P) =160 Hz), 58.3, 58.7 (2-CH), 120.1, 125.0, 129.4, 149.5 (Cₐᵣₒₘ), 167.3, 168.4 (CO); [α]D²⁰=25.9° (C₁, CHCl₃); MS (FAB+) m/z 401 (M+H)⁺.

Di(3-acetamidophenyl) 1-((S)-prolyl)pyrrolidine-2(R,S)-phosphonate hydrochloride (11d).

B. Yield 83%: solid foam; ¹H-NMR (DMSO) δ (ppm) 1.6-2.6 (m, 8H, 3-CH₂ and 4-CH₂), 2.1 (s, 6H, COCH₃), 3.3-3.8 (m, 4H, 5-CH₂), 4.7 (m, ¹H, 2-CH), 4.9 (m, ¹H, 2-CH), 6.7-7.6 (m, 8Hₐᵣₒₘ), 8.5 (m, ¹H, N+H), 9.7 (m, ¹H, N+H), 9.9 (m, 2H, CONHPh); Anal. (C₂₅H₃₁N₄O₆P 1.5 HCl 0.5 H₂O) C, H, N.

Di(4-acetamidophenyl) 1-((S)-prolyl)pyrrolidine-2(R,S)-phosphonate hydrochloride (11e).

B. Yield 90%: solid foam; ¹H-NMR (DMSO) δ (ppm) 1.6-2.6 (m, 8H, 3-CH₂ and 4-CH₂), 2.1 (s, 6H, COCH₃), 3.3-3.8 (m, 4H, 5-CH₂), 4.6 (m, ¹H, 2-CH), 4.9 (m, ¹H, 2-CH), 7.1 (m, 4Hₐᵣₒₘ), 7.6 (m, 4Hₐᵣₒₘ), 8.7 (m, ¹H, N+H), 10.0 (m, 2 H, CONHPh), 10.2 (m, ¹H, N+H); 31P-NMR (DMSO) δ (ppm) 17.48, 17.62; Anal. (C₂₅H₃₁N₄O₆P HCl 2 H₂O) C, H, N.

Di(4-methylsulfonylaminophenyl) 1-((S)-prolyl)-pyrrolidine-2(R,S)-phosphonate hydrochloride (11f).

B. Yield 80%: solid foam; ¹H-NMR (DMSO) δ (ppm) 1.55-2.5 (m, 8H, 3-CH₂ and 4-CH₂), 2.95 (s, 6H, SO₂CH₃), 3.3-3.8 (m, 4H, 5-CH₂), 4.55 (m, ¹H, 2-CH), 4.85 (m, ¹H, 2-CH), 7.15 (m, 8Hₐᵣₒₘ), 8.6 (br s, ¹H, N+H), 9.79 (s, 2H, SO₂NHPh), 10.3 (br s, ¹H, N+H); Anal. (C₂₃H₃₁N₄O₈PS₂ HCl) C, H, N.

Di(3-ureylphenyl) 1-((S)-prolyl)pyrrolidine-2(R,S)-phosphonate hydrochloride (11g).

B. Yield 90%: solid foam; ¹H-NMR (CD3OD) δ (ppm) 1.4-2.5 (m, 8H, 3-CH₂ and 4-CH₂), 3.3-3.8 (m, 4H, 5-CH₂), 4.5 (m, ¹H, 2-CH), 4.8 (m, ¹H, 2-CH), 6.4-7.2 (m, 6Hₐᵣₒₘ), 7.4 (m, 2Hₐᵣₒₘ); MS (FAB+) m/z 517 (M+H)⁺; Anal (C₂₃H₂₉N₆O₆P HCl 2.3 H₂O) C, H, N.

Di[4-(N-benzoylglycylamino)phenyl]-1-((S)-prolyl)pyrrolidine-2(R,S)-phosphonate hydrochloride (11h).

B. Yield 87%; solid foam; ¹H-NMR (DMSO) δ (ppm) 1.6-2.6 (m, 8H, 3-CH₂ and 4-CH₂), 3.2-3.6 (m, 4H, 5-CH₂), 4.2 (d, 4H, NCH₂CO), 4.5-4.9 (m, 2H, 2-CH), 6.9-7.9 (m, 20H, 18Hₐᵣₒₘ and NH), 8.5 (m, ¹H, N+H), 10.0 (s, 2H, NH), 10.2 (m, ¹H, N+H); MS (FAB+) m/z 753 (M+H)⁺; Anal. (C₃₉H₄₁N₆O₈P HCl 2 H₂O) C, H, N.

Di[4-(N-glycylamino)phenyl]-1-(S)-prolyl)pyrrolidine-2(R,S)-phosphonate trihydrochloride (11i).

B. Yield 81%: solid foam; ¹H-NMR (DMSO) δ (ppm) 1.55-2.5 (m, 8H, 3-CH₂ and 4-CH₂), 3.3-3.8 (m, 4H, 5-CH₂), 3.8 (s, 4H, COCH₂N), 4.55 (m, ¹H, 2-CH), 4.85 (m, ¹H, 2-CH), 7.15 (m, 4Hₐᵣₒₘ), 7.65 (m, ⁴Hₐᵣₒₘ) 8.35 (br s, 6H, N+H), 8.5 (br s, ¹H, N+H), 10.35 (br s, ¹H, N+H), 11.0 (s, 2H, NH); Anal. (C₂₅H₃₃N₆O₆P 3 HCl 2.5 H₂O) C, H, N.

Di (4-(S)-alanylaminophenyl)-1-((S)-prolyl)pyrrolidine-2(R,S)-phosphonate trihydrochloride (11j).

B. Yield 70%; solid foam; ¹H-NMR (D2O) δ (ppm) 1.49 (d, 6H, CH₃), 1.65-2.60 (m, 8H, 3-CH₂ and 4-CH₂), 3.23-3.75 (m, 4H, 5-CH₂), 4.12 (m, 2H, CH of Ala), 7.05 (m, 4H, Hₐᵣₒₘ), 7.35 (m, 4H, Hₐᵣₒₘ); Anal. (C₂₇H₃₇N₆O₆P 3 HCl 0.5 H₂O) C, H; N: calcd, 12.16; found, 11.73.

Di(4-(S)-pyroglutamylaminophenyl)-1-((S)-prolyl)pyrrolidine-2(R,S)-phosphonate hydrochloride (11k).

B. Yield 95%: solid foam; ¹H-NMR (DMSO) δ (ppm) 1.5-2.4 (m, 16H, 3-CH₂ and 4-CH₂), 3.1-4.2 (m, 6H, 5-CH₂ and 5-CH), 4.5 (m, ¹H, 2-CH Pro), 4.85 (m, ¹H, 2-CH ProP), 7.1 (m, 4Hₐᵣₒₘ), 7.65 (m, 4Hₐᵣₒₘ), 7.9 (s, 2H, NH), 8.6 (m, ¹H, N+H), 10.1 (m, ¹H, N+H), 10.3 (s, 2H, NH); Anal. (C₃₁H₃₇N₆O₈P 1.5 HCl 3 H₂O) C, H, N.

Di{4-[-(S)-(2-methoxycarbonyl-2-acetamido)ethyl]phenyl}1-((S)-prolyl)pyrrolidine-2-phosphonate hydrochloride (11l).

B. Yield 85%: solid foam; ¹H-NMR (DMSO) δ (ppm) 1.6-2.6 (m, 8H, 3-CH₂ and 4-CH₂), 1.9 (s, 6H, COCH₃), 3.0 (d, 4H, CH₂Ar), 3.2-3.8 (m, 4H, 5-CH₂), 3.7 (s, 6H, COOCH₃), 4.4-4.9 (m, 4H, 2-CH), 7.1 (m, ⁸Hₐᵣₒₘ), 8.2 (d, 2H, AcNH), 8.7 (m, ¹H, N+H), 10.4 (m, ¹H, N+H); MS (FAB+) m/z 687 (M+H)⁺; Anal. (C₃₃H₄₃N₄O₁₀P 1.5 HCl 2 H₂O) C, N; H: calcd, 6.29; found, 5.81.

Di{4-[(ethoxycarbonyl)methylaminocarbonyl]phenyl}1-((S)-prolyl)pyrrolidine-2(R,S)-phosphonate (11n).

B. Yield 86%: solid foam; ¹H-NMR (DMSO) δ (ppm) 1.3 (t, 6H, CH₃), 1.4-2.6 (n, 8H, 3-CH₂ and 4-CH₂), 3.2-3.8 (m, 4H, 5-CH₂), 4.0 (m, 4H, NCH₂CO), 4.2 (q, 4H, OCH₂), 4.6 (m, ¹H, 2-CH), 4.9 (m, ¹H, 2-CH), 7.3 (m, 4Hₐᵣₒₘ), 7.9 (m, 4Hₐᵣₒₘ), 8.7 (m, 4H, N+H and NH); MS (FAB+) m/z 659 (M+H)⁺; Anal. (C₃₁H₃₉N₄O₁₀P HCl 1.5 H₂O) C, H, N.

Di{4-[2-(methoxycarbonyl)ethylaminocarbonyl]phenyl}1-((S)-prolyl)-pyrrolidine-2(R,S)-phosphonate hydrochloride (11o).

B. Yield 79%: solid foam; ¹H-NMR (DMSO) δ (ppm) 1.2-2.8 (m, 8H, 3-CH₂ and 4-CH₂), 2.65 (t, 4H, CH₂COO), 3.2-3.9 (m, 8H, 5-CH₂ and CH₂N), 3.8 (s, 6H, COOCH₃), 4.7 (m, ¹H, 2-CH), 4.9 (m, ¹H, 2-CH), 7.55 (m, 4Hₐᵣₒₘ), 8.15 (m, 4Hₐᵣₒₘ), 8.55 (m, 4H, N+H and NH).

Di[4-(n-propylaminocarbonyl)phenyl]1-((S)-prolyl)-pyrrolidine-2(R,S)-phosphonate hydrochloride (11p).

B. Yield 80%: solid foam; ¹H-NMR (CD3OD) δ (ppm) 0.95 (t, 6H, CH₃), 1.6 (m, 4H, CH₂), 1.7-2.6 (m, 8H, 3-CH₂ and 4-CH₂), 3.2-3.9 (m, 8H, 5-CH₂ and CH₂N), 4.65 (m, ¹H, 2-CH Pro), 5.1 (m, ¹H, 2-CH ProP), 7.3 (m, ⁴Hₐᵣₒₘ), 7.85 (m, 4Hₐᵣₒₘ); ¹³C-NMR (CD3OD) δ (ppm) 11.7 (CH₃), 23.5 (CH₂), 25.0 (4-CH₂), 25.8 (4-CH₂), 27.3 (3-CH₂), 29.7 (3-CH₂), 42.8 (NCH₂), 47.4 (5-CH₂), 48.3 (5-CH₂), 55.9 (d, 2-CH-P, 1J (C-P) =161 Hz), 60.6 (2-CH), 121.4, 130.4, 133.1, 153.7 (Cₐᵣₒₘ), 168.8 (CO), 169.2 (CO).

2,2′-Biphenylacetyl phosphite (16).

A solution of chloroanhydride 15 (0.152 mol, 38 g) in dry THF (50 mL) was added dropwise to a solution of ACOH (0.152 mol, 9.1 mL) and Et₃N (0.152 mol, 21.3 mL) in dry THF (100 mL) with stirring at 0° C. The cooling was removed and the mixture was stirred at room temperature for 3 h. The solid was filtered off and the filtrate was evaporated. The crude product was used in the next step without further purification. Yield 40 g (96%): viscous oil; ¹H-NMR (CDCl₃) δ (ppm) 2.1 (s, 3H, CH₃CO), 6.9-7.5 (m, 8H, Hₐᵣₒₘ).

2,2′-Biphenyl 1-(benzyloxycarbonyl-(S)-prolyl)-pyrrolidine-2(R,S)-phosphonate (17a).

Prepared according to the general procedure for diaryl phosphonates with acetal 6a and phosphite 16. Yield 27%: solid foam; ¹H-NMR (CDCl₃) δ (ppm) 1.2-2.9 (m, 8H, 3-CH₂ and 4-CH₂), 3.1-4.0 (m, 4H, 5-CH₂), 4.55 (m, ¹H, 2-CH Pro), 4.85 (m, H, 2-CH ProP), 5.2 (br s, 2H, CH₂Ph), 7.1-7.85 (m, 15Hₐᵣₒₘ); MS (EI) m/z (relative intensity) 532 (M, 40), 397 (20), 300 (26), 232 (29), 215 (41), 204 (45), 168 (42), 160 (74), 91 (100), 70 (48).

2,2′-Biphenyl 1-(t-butyloxycarbonyl-(S)-prolyl)-pyrrolidine-2(R,S)-phosphonate (17b).

Prepared according to the general procedure for diaryl phosphonates with acetal 6b and phosphite 16.

Yield 24%: solid foam; ¹H-NMR (CDCl₃) δ (ppm) 1.3-2.55 (m, 8H, 3-CH₂ and 4-CH₂), 1.5 (s, 9H, CH₃), 3.35-3.9 (m, 4H, 5-CH₂), 4.55 (m, 1H, 2-CH), 4.9 (m, ¹H, 2-CH), 6.95-7.75 (m, 8H, Hₐᵣₒₘ); MS (FAB+) m/z 499 (M+H)⁺.

2-(2′-Hydroxyphenyl)phenyl methyl 1-(S)-prolyl)-pyrrolidine-2(R,S)-phosphonate hydrochloride (18).

Prepared from the Z-protected derivative 17a using procedure “B” for diaryl phosphonates.

Yield 92%: solid foam; ¹H-NMR (CDCl₃) δ (ppm) 1.5-2.6 (m, 8H, 3-CH₂ and 4-CH₂), 3.1-3.9 (m, 7H, 5-CH₂ and OCH₃), 4.4-4.8 (m, 2H, 2-CH Pro and 2-CH ProP), 6.85 (br S, ¹H, PhOH), 7.1-7.6 (m, 8Hₐᵣₒₘ) 8.4 (m, ¹H, N+H), 9.6 (m, ¹H, N+H); ¹³C-NMR (CDCl₃) δ (ppm) 25.7 (4-CH₂), 26.1 (4-CH₂), 28.2 (3-CH₂), 30.5 (3-CH₂), 48.1 (5-CH₂), 48.6 (5-CH₂), 55.6 (OCH₃), 55.6 (2-CH-P, 1J (C-P)=142 Hz), 60.8 (2-CH), 117.8, 121.0, 126.3, 130.1, 132.5, 133.8 149.7, 156.2 (Cₐᵣₒₘ), 168.9 (CO); MS (FAB+) m/z 431 (M+H)⁺; Anal. (C₂₂H₂₇N₂O₅P HCl H₂O) C, H; N: calcd, 5.78; found, 6.26.

2,2′-Biphenyl 1-((S)-prolyl)pyrrolidine-2(R,S)-phosphonate hydrochloride (19).

Prepared from the Boc-protected derivative 17b using procedure “A” for diaryl phosphonates.

Yield 81%: solid foam; ¹H-NMR (CDCl₃) δ (ppm) 1.3-2.55 (m, 8H, 3-CH₂ and 4-CH₂), 3.35-3.75 (m, 4H, 5-CH₂), 4.75 (m, 2H, 2-CH), 7.0-7.5 (m, 8H, Hₐᵣₒₘ) 10.9 (m, 2H, N+H); ¹³C-NMR (CDCl₃) δ (ppm) 24.4 (4-CH₂), 24.9 (4-CH₂), 26.7 (3-CH₂), 28.7 (3-CH₂), 46.6 (5-CH₂), 51.9 (5-CH₂), 53.4 (2-CH-P, 1J (C-P) =151 Hz), 59.2 (2-CH), 116.9, 120.9, 121.7, 121.9, 125.8, 126.6, 128.4, 129.0, 130.3, 131.5, 147.9 (Cₐᵣₒₘ), 167.8 (CO); MS (FAB+) m/z 399 (M+H)⁺; Anal. (C₂₁H₂₃N₂O₄P 1.4 HCl 1.1 H₂O) C, H; N: calcd, 5.97; found, 5.43.

### EXAMPLE 2

Synthesis of a Series of Diaryl Phosphonates with an Alanine as Residue aa

Diphenyl alanine phosphonates were synthesized following the principle described by Oleksyszyn et al., Synthesis 1979, 985-986. The method was used with acetaldehyde for the preparation of the alanine analogue, diphenyl 1-benzyloxycarbonylaminoethane phosphonate. After deprotection, the mixed anhydride method was used to couple diphenyl 1-aminoethane phosphonate to various amino acids:glycine, L-alanine, L-valine, L-isoleucine, L-phenylalanine and L-proline.

Experimental

Preparation of the Diphenyl Alanine Phosphonates

Diphenyl 1-(N-benzyloxycarbonyl) aminoethane phosphonate (X)

A mixture of triphenyl phosphate (100 mmol, 31.03 g), acetaldehyde (150 mmol, 6.61 g—freshly distilled), benzylcarbamate (100 mmol, 15.10 g) and glacial acetic acid (15 ml) was stirred for about 30 minutes until the exothermic reaction subsides.

The mixture was heated at 80-85° C. for one hour and the volatile products were removed on a rotary evaporator under reduced pressure with heating on a boiling water bath. The oily residue was dissolved in methanol (180 ml) and left overnight for crystallization at −10° C. The crystalline ester was collected by filtration, redissolved in a small amount of hot chloroform (30-40 ml) and recrystallization occurred by adding a 4-fold volume of methanol.

Yield: 46%; m.p.: 117-118° C. IR (KBr): 3260 (NH); 3050, 3020 (CHar); 2960, 2920 (CHalif); 1720 (C═O) 1240 (P═O); 1200 (P—O); 740, 690 (C₆H₅); ¹H-NMR (CDCl₃, 60 MHz): 1.55 (3H, dd, J_{PH}=17.5 Hz, J_{HH}=7 Hz, CH₃)4.53 (1H, m, CH); 5.10 (2H, s, OCH,); 5.37 (1H, br, NH); 6.93-7.23 (10H, m, Har); 7.23-7.37 (5H, m, Har)

Diphenyl 1-aminoethane phosphonate hydrobromide (Y)

Compound X (10 mmol, 4.11 g) was dissolved in a 45% solution of hydrogen bromide in acetic acid (5 ml). After-one hour at room temperature the solvent and volatile products were removed under reduced pressure on a boiling water bath. The oily residue was triturated with dry ether. The crystals were filtered and dried over sodium hydroxide in a vacuum oven to give the pure hydrobromides.

Yield: 95%; m.p.: 148.5° C.; IR (KBr): 3100-2700 (NH₃⁺); 1230 (P=O); 1200 (P-0) 760, 680 (C₆H₅); ¹H-NMR (CDCl₃+d6-DMSO, 60 MHz): 1.77 (3H, dd, J_{PH}=17.0 Hz, J_{HH}=7 Hz, CH₃); 4.05 (1H, m, CH); 6.97-7.30 (1OH, m, Har)

Diphenyl 1(R,S)-[N-benzyloxycarbonyl-L-phenylalanyl]-aminoethane phosphonate, Diphenyl 1(R,S)-[N-benzyl-oxycarbonyl-L-prolyl]aminoethane phosphonate (T, Q)

General procedure for the mixed anhydride coupling with isobutyl chloroformate.

The Z-protected amino acid (5 mmol) was dissolved in dry tetrahydrofuran (25 ml) and cooled to −150° C. in an ice-sodium chloride bath. N-Methyl-morpholine (5 mmol, 0.51 g) and isobutyl chloroformate (5 mmol, 0.68 g) were added to this solution and stirring was continued for 10 minutes. A solution of Y (5 mmol, 1.79 g) and triethylamine (5 mmol, 0.51 g) in dimethylformamide (10 ml), was introduced dropwise to the stirring mixture, keeping the temperature below −100° C.

After one hour in a cold water bath, the reaction mixture was allowed to warm to room temperature. The hydrochlorides of N-methylmorpholine and triethylamine were removed by filtration and washed with tetrahydrofuran. The combined filtrate and washings were concentrated on a rotary evaporator. The residue was dissolved in ethyl acetate (75 ml) and water (25 ml) and the organic phase was washed with 5% sodium bicarbonate (25 ml), 0.5N hydrogen chloride solution (25 ml) and saturated sodium chloride solution (25 ml), dried over sodium sulphate and evaporated to dryness under reduced pressure.

T: Yield: 84%; oil; IR(KBr):3280(NH); 3060(CHar); 2960, 2930, 2880 (CHalif); 1720-1660 (C=O); 1250(P=O) 1180(P-0);760, 685(C₆H₅)

¹H-NMR (CDCl₃, 60 MHz): 1.33 (3H, dd, J_{PH}=17.5 Hz, J_{HH}=7.0 HZ, CH₃); 2.80-3.10 (2H, m, βH); 4.27-4.73 (2H, m, αH, CH); 5.05 (2H, m, CH₂); 5.53 (2H, br, NH); 6.87-7.30 (20H, m, Har)

Q: Yield: 79%; oil; IR(KBr):3280(NH); 3060(CHar); 2960, 2930, 2880 (CHalif); 1720-1660 (C=O);1260 (P=O);1180(P-O); 760, 685 (C₆H₅)

¹H-NMR (CDCl₃, 60 MHz): 1.43 (3H, dd, J_{PH}=18.0 Hz, J_{HH}=7.0 Hz, CH₃); 1.75-2.10 (4H, m, βH, yH); 3.35-3.70 (2H, m, δH); 4.25-4.50 (1H, m, αH); 4.85 (1H, m, CH); 5.13 (2H, s, CH₂); 7.10-7.4-0 (15H, m, Har)

Diphenyl 1(R,S)-L-phenylalanyl aminoethane phosphonate, diphenyl 1(R,S)-L-prolyl aminoethane phosphonate (K, L) General procedure for removal of the Z-group by hydrogenolysis

The Z-protected dipeptide phosphonates T and Q (1.5 mmol) were dissolved in dry methanol (20 ml) and acetic acid (0.7 ml). After the addition of palladium on charcoal (10%, 0.25 g), the air above the solution was displaced with nitrogen and hydrogen was led over the solution for 3 hours. Before removing the solvent under reduced pressure, the flask was flushed with nitrogen for 10 minutes. The residue was dissolved in dry chloroform (15 ml) and 0.5N hydrogen chloride in ethyl acetate was added slowly to the stirring solution. Removal of the solvent under reduced pressure and co-evaporation with 0.5N hydrogen chloride in ethyl acetate (10 ml) afforded a white foam, which was triturated with dry ether. After 2 days at −10° C., the crystals were filtered off and the hygroscopic product was kept in a desiccator.

K: Yield: 80%; oil; IR: 3240(NH);3100-2800(NH₃⁺); 1690 (C=O); 1250 (P=O); 1180 (P-O); 750,680 (C₆H₅)

¹H-NMR (CDCl₃, 250 MHz): 1.15-1.50 (3H, dd, J_{PH}=17 Hz, J_{HH}=6.7 Hz, CH₃); 3.16, 3.43 (2H, m, H); 44.46-4.58; 4.46-4.70 (3H, m, αH, CH, NH); 6.98-7.34 (15H, m, Har); 8.30-8.52 (2H, -br, NH₂)

L: Yield 751; oil; IR:3240(NH):3100-2800 (NH₃⁺); 1690(C=O);1250(P=O); 1180(P-O); 760, 680(C₆H₅)

¹H-NMR (CDCl₃, d6-DMSO, 250 MHz): 1.37-1.65 (3H, m, CH₃); 1.77-2.03 (4H, m, βH, yH); 3.38-3.51 (2H, m, δH); 4.31 (1H, m, NH); 4.55 (1H, m, αH); 4.77-4.88 (1H, m, CH); 7.13-7.31 (10H, m, Har)

### EXAMPLE 3

DPP IV Inhibition and Stability of the Inhibitors

In a previous study (Lambeir, A. M. et al. Dipeptide-derived Diphenyl Phosphonate Esters: Mechanism-based Inhibitors of Dipeptidyl Peptidase IV. Biochim. Biophys. Acta 1996, 1290, 76-82) on the role of the P-2 amino acid in dipeptide diphenyl phosphonates, the present inventors showed that proline in this position gives one of the most potent inhibitors. A major advantage of 5 (the mixture of diastereoisomners of 11a) was its greater stability in human citrated plasma compared to the other dipeptide derivatives. The stability of 5 in plasma equals the stability in buffer, whereas for the other compounds the stability in plasma was reduced compared to buffer. This reflects the relative stability of a Pro-Pro amino acid sequence to proteolytic breakdown, and indicates that the decrease in activity of 5 is mainly caused by hydrolysis of the phosphonate ester.

To increase the inhibitory activity while retaining the stability several substituents were introduced on the phenyl rings that act as leaving groups.

The influence of electron-donating or -withdrawing substituents on enzyme inhibition and stability was investigated as shown in Table 1. All compounds were irreversible inhibitors of DPP IV, probably due to the formation of a phosphonylated serine at the active site of the enzyme. The inactivation rate constants were calculated from experimental IC₅₀ values and were in reasonable agreement with the measured inactivation rate constants, where available.

A good correlation was observed between the electron-withdrawing properties of the substituent and its activity (FIG. 1). Introduction of an electron-donating substituent (4—OH, 11c) decreases potency, whereas an electron-withdrawing substituent (4-methoxycarbonyl, 11m) increases potency.

Quite unexpectedly, a striking divergence was observed in the correlation between the Hammett constant and the inhibitory activity for compounds 11e (4-acetylaminophenyl) and 11f (4-methylsulfonylaminophenyl). Both the compounds (11e) and (11f) strongly inhibited DPP IV. These compounds have similar Hammett constants compared to the diastereoisomeric mixture of 5, but are nevertheless about 100 times more potent.

Introduction of other acylamino substituents was investigated. The 4-glycylamino (11i) and the 4-alanylamino (11j) have a comparable activity, but are considerably less stable in plasma than the 4-acetylamino (11e) substituted diphenyl phosphonate.

The half-life of the inhibitors seems to also correlate with the Hammett constant (FIG. 2). Compounds with similar electronic properties (e.g. 5 and 11e) have a similar stability in plasma.

The O-methyl derivative (18) is almost as potent and stable as 5.

The correlation between electronic properties and inhibitory activity and between electronic properties and stability results in an inverse correlation between inhibitory activity and stability (FIG. 3). This means that a more active compound is also more unstable. Therefore, the higher potency (IC₅₀=0.4 μM) and equal stability (t_{½}=320 min) of the paracetamol substituted phenyl phosphonate (11e) is a major improvement to the unsubstituted 5.

Experimental:

DPP IV was purified from human seminal plasma as described previously (De Meester et al., J. Immunol. Meth. 189, 99-105 (1996)). Enzymatic activity was measured at 37° C. in a Spectramax 340 (Molecular Devices) microtiterplate reader using Gly—Pro-p-nitroanilide (Sigma) as a chromogenic substrate. The reaction was monitored at 405 nm and the initial rate was determined between 0 and 0.25 absorbance units. The reaction mixture contained 2 mM substrate, approximately 1 mU of DPP IV, 40 EM TRIS-HCl buffer, pH 8.3, and a suitable amount of inhibitor (ranging between 0 an 10 mM) in a total volume of 0.2 ml. Activity measurements were routinely performed in duplicate.

The IC₅₀ value is defined as the concentration of inhibitor required to reduce the DPP IV-activity to 50% after a 15 min pre-incubation with the enzyme at 37° C. before addition of the substrate. Inhibitor stock solutions (100 mM) were prepared in DMSO or phosphate buffer, pH 7.4, depending on the solubility of the compound, and stored at −20° C. Stock solutions were diluted with 50 mM TRIS-HCl buffer, pH 8.3, as required, immediately before the experiment. Since the compounds described in this paper completely inactivate DPP IV following pseudo-first order kinetics, the IC₅₀ value is inversely correlated with the second order rate constant of inactivation (Lambeir et al., (1996), supra). For a simple pseudo-first order inactivation process, the activity after incubation with inhibitor (vi) varies with the inhibitor concentration (i) as described in the following equation: vi=vo * e-k.i.t, where vo is the activity in absence of inhibitor, k is the second order rate constant of inactivation and t is the time. Since at i=IC₅₀ by definition vi=½ vo, it follows from the equation that k=ln2/(ti*IC₅₀) where ti=15 min. These calculated k values are listed in Table 1.

TABLE 1
Potency and stability of DPP IV inhibitors

| | | Configuration | IC₅₀ | k_{calc} | t_{1/2} (min) |
|---|---|---|---|---|---|
| | R₁ | at Pro^{P}C(2) | (μM) | (M⁻¹ s⁻¹) | In plasma |
| 5 | H | R,S | 32 (n = 1) | 2.4*10¹ | 250 ± 10 |
| 11a | H | R | 15 ± 3 (n = 3) | 5.1*10¹ | 300 ± 30 |
| 11a | H | S | >10⁴ | | |
| 11d | 3-AcNH | R,S | 0.8 ± 0.1 (n = 2) | 9.6*10² | 220 ± 30 |
| 11e | 4-AcNH | R,S | 0.4 ± 0.2 (n = 5) | 1.9*10³ | 320 ± 140 |
| 11f | 4-MeSO₂NH | R,S | 0.40 ± 0.02 (n = 2) | 1.9*10³ | 150 ± 30 |
| 11g | 3-H₂NCONH | R,S | 2.3 ± 0.3 (n = 2) | 3.3*10² | 210 ± 80 |
| 11h | 4-(N-Bz-Gly-NH) | R,S | 0.7 ± 0.3 (n = 2) | 1.1*10³ | 93 ± 3 |
| 11i | 4-(H-Gly-NH) | R,S | 0.5 ± 0.1 (n = 2) | 1.5*10³ | 28 ± 3 |
| 11j | 4-(H-(S)-Ala-NH) | R,S | 0.6 ± 0.2 (n = 2) | 1.3*10³ | 8 ± 1 |
| 11k | 4-((S)-Pyr-NH) | R,S | 5.0 ± 0.8 (n = 2) | 1.5*10² | 170 ± 30 |
| 11l | 4-[(2S)-MeO₂CCH(NHAc)CH₂] | R,S | 1.4 ± 0.5 (n = 4) | 5.5*10² | 190 ± 150 |
| 11m | 4-MeO₂C | R,S | 0.016 ± 0.004 (n = 2) | 4.8*10⁴ | 19 ± 1 |
| 11n | 4-(ErO₂CCH₂NHCO) | R,S | 0.023 ± 0.007 (n = 2) | 3.3*10⁴ | 35 ± 2 |
| 11o | 4-[MeO₂C(CH₂)₂NHCO] | R,S | 0.036 ± 0.006 (n = 4) | 2.1*10⁴ | 26 ± 1 |
| 11p | 4-[CH₃(CH₂)₂NHCO] | R,S | 0.03 ± 0.01 (n = 2) | 2.6*10⁴ | 12 ± 1 |
| 18 | P(OMe)(OC₆H₄(2-OH-C₆H₄)) | R,S | 47 ± 18 (n = 2) | 1.6*10¹ | 140 ± 80 |
| 19 | P-2,2′-biphenyl | R,S | 31 ± 6 (n = 2) | 2.5*10¹ | 6 ± 1 |

IC₅₀ values were determined after 15 min pre-incubation with DPP IV at 37° C. The listed values are the average of n independent measurements ± the standard deviation. Functional stability in plasma was estimated by fitting the inverse of the apparent IC₅₀ values versus time with a single exponential decay. The half-life is listed ± the standard error of fit.

For some inhibitors (5, 11a, 11b, 11d, 11e, 11h, 11n) the inactivation rate constant was determined from the time course of inhibition as described before (Lambeir et al., (1996), supra).

The functional stability of the inhibitors was estimated by measuring the inhibitory potency (apparent IC₅₀) of a 1 mM dilution of the compounds in citrated human plasma at 3 or 4 time points between 0 and 300 min at 37° C. Fitting the inverse of the apparent IC₅₀ values versus time with a single exponential decay gives the half-lives reported in table 1.

### EXAMPLE 4

Inhibition of Prolyl Oligopeptidase (PO) by Protected Prolylpyrrolidine Diaryl Phosphonates

The inhibitory capacity of various compounds of the invention was evaluated. The results are shown in table 2.

TABLE 2
Potency of the compounds 10 as PO inhibitors. IC₅₀ values
are expressed in μM

| compound | R1 or R2 | IC₅₀ |
|---|---|---|
| 10d | 3-AcNH | 10 |
| 10e | 4-AcNH | 21 |
| 10f | 4-MeSO₂NH | 10 |
| 10h | 4-(N-Bz-Gly-NH) | 12 |
| 10i | 4-(N-Z-Gly-NH) | 15 |
| 10j | 4-(N-Z-Ala-NH) | >500 |
| 10l | 4-[(2S)-MeO₂CCH—(NHAc)CH₂] | 20 |
| 10k | 4-((S)-Pyr-NH | 9 |
| 10n | 4-(EtO₂CCH₂NHCO) | 5 |
| 10o | 4-[MeO₂C—(CH₂)₂NHCO] | 2.8 |
| 10p | 4-[CH₃(CH₂)₂NHCO] | 3.2 |

Experimental

Prolyl oligopeptidase was purified from human platelets and the enzyme activity was measured using Z—Gly—Pro—AMC (4.4 mM) as the substrate in a K-phosphate buffer 100 mM, pH 7.5 containing 1 EM EDTA, 1 mM dithiothreitol and 1 mM NaN₃. The incubation was carried out during 20 min at 37° C. The reaction was stopped by the addition of 5 volumes 1.5 M acetic acid. Fluorescence was measured at 370 and 440 nm as excitation and emission wavelengths respectively. The inhibitors were added at concentrations varying between 1 μM and 1 mM.

### EXAMPLE 5

Inhibition of Dipeptidyl Peptidase II by Dipeptide-derived Diaryl Phosponate Esters With an ala in Position aa

The compounds Pro-AlaP(OPh)2 and Phe-AlaP(OPh)2 inhibited DPP II in vitro and the IC₅₀ values calculated were 1.5 mM for Pro-AlaP(OPh)2 and 0.8 mM for Phe-AlaP(OPh)2.

Experimental:

Dipeptidyl peptidase II was semi-purified from rabbit kidney and its activity was determined by the hydrolysis of Lys—Ala—4—MeO—2—NA 1.4 mM (Sigma, L-2270) in 100 mM acetate buffer, pH 5.5 containing 2 mM EDTA. After incubation during 20 minutes at 37° C., the reaction was stopped by the addition of 10 fold excess sodium-acetate pH 3.6. The fluorescence of the formed 4-MeO-2-NA was measured at 340 and 425 nm as excitation and emission wavelengths respectively. For the determination of the IC₅₀ values the compounds were tested at a series of concentrations ranging from 1 μM to 5 mM during incubation.

### EXAMPLE 6

In vitro Cytotoxicity and Efficacy of Prolylpyrrolidine Diaryl Phosphonates in Human Peripheral Blood Mononuclear Cells (PBMC)

Based on inhibition potency, stability in plasma and synthesis efficiency, compounds 11e and 11n were selected for further in vitro and in vivo studies. Both compounds were evaluated in human peripheral blood mononuclear cells (PBMC) and did not show cytotoxicity in freshly isolated mononuclear cells or phytohemagglutinin stimulated blasts when concentrations up to 100 μM were used. Under these circumstances, more than 90% of the DPP IV activity in cell lysates as well as in supernatants was inhibited. This is in contrast with the results obtained for the active diastereoisomer of the unsubstituted diphenyl phosphonate (11a), where no satisfactory inhibition of DPP IV activity could be reached without cytotoxic effects on PBMC cultures. The compounds 11e and 11n are therefore promising tools for further studies on cellular level. The irreversible mechanism of inhibition overcomes the rather limited stability of the compounds in biological media.

Experimental:

Peripheral blood mononuclear cells were isolated from buffy coats (obtained from the blood transfusion center of Antwerp). After dilution (¼) in phosphate buffer saline (PBS), cells were layered onto Ficoll-Hypaque density gradient (Pharmacia, Uppsala, Sweden) and centrifuged at room temperature at 550×g during 20 min. The interfaces were collected and washed 3 times in RPMI-1640. Finally the cells were resuspended at 1×10⁶ cells/mL in RPMI-1640 containing 10% heat-inactivated foetal calf serum, and antibiotics (penicillin/streptomycin) (Gibco). These freshly isolated cells were used immediately for inhibitor studies or first stimulated with phytohaemagglutinin (Murex diagnostics) at 1 μg/mL during 3 days at 37° C. in a 5% CO₂ humidified incubator. Inhibitor (stock solution at −80° C. in phosphate buffer, diluted ex tempore in RPMI-1640) or vehicle alone was added to the cells (5×10⁶/test) at different concentrations. After overnight incubation at 37° C., an aliquot was taken for cytotoxicity evaluation by 0.4% trypan blue exclusion. The remaining cells were washed 3 times in PBS and the final cell pellet was solubilised in 200 μL PBS containing 1% v/v Triton X-100 and 100 KIU/mL aprotinin (Bayer) and centrifuged during 10 min at 20000×g. Supernatants were used immediately for enzyme assay and protein determination by the Bradford micro-assay. Specific activities (U/g protein) are compared and the % inhibition is given toward control samples without inhibitor.

### EXAMPLE 7

DPP IV Inhibition in Vivo

It was reported previously (De Meester et al., Biochem. Pharmacol. 54, 173-179 (1997)) that a single intravenous injection of 5 (0.3-5 mg/kg) in rabbits caused a decrease in plasma DPP IV activity with more than 80% and it took more than 20 days for complete recovery.

In rats, a comparable dose per weight intravenously did not result in a sufficient inhibition of circulating DPP IV, because adequate inhibition could not be reached without severe systemic toxicity. However, a combination of subcutaneous and intraperitoneal injections of 11a allowed us to bring plasma DPP IV to less than 15% of pre-treatment values. The observation that monotherapy with the diastereoisomeric mixture of 11a (5) not only significantly prolonged graft acceptance upon alloantigen challenge, but occasionally also caused systemic toxicity and more often local ulcerations, stimulated the in vivo testing of 11e and 11n in rabbits, rats and mice.

In rabbits, the higher in vivo potency of 11n compared to 11e was also observed. The IC₅₀ values for inhibition of plasma DPP IV in rabbits upon single intravenous injection was below 20 μg/kg for 11n and was around 0.2 mg/kg for 11e. In this species single intravenous injection of 0.2 mg/kg of 11n inhibited plasma DPP IV activity for more than 90% during at least 24 h, without side effects (see FIG. 4).

In rats, 11e as well as 11n could keep plasma DPP IV activity below 10% of pre-treatment values by daily subcutaneous injection of 50 mg/kg (initial dose 100 mg/kg), without any sign of acute systemic or local toxicity. FIG. 5 depicts residual plasma DPP IV activity in rats treated subcutaneously on days 0 to 5 with 11e.

Different administration routes in rats were examined for compound 11n: oral as well as subcutaneous as intraperitoneal and intrarectal routes of administration allowed an in vivo inhibition of DPP IV. The intraperitoneal route being the least efficient i.e. no 50% inhibition after 24 h while for the other routes DPP IV activity was still very low (<35% of initial value) 1 day after a single dose (all routes 50 mg/kg).

In mice, a pharmacologically useful inhibition was only obtained with 11n and not with 11a and 11e. The molecular basis for the large interspecies differences in efficiency remains to be elucidated.

Experimental:

Male New Zealand white rabbits (2.5-3.5 kg), Wistar rats (250-350 g) and Swiss mice (23-36 g) were allowed to adjust to their environment for at least 7 days. They received standard diet and water ad lib. Test compounds were dissolved in 50 mM phosphate buffer pH 7.4 at concentrations ranging from 10-100 mg/mL and stored in aliquots at −80° C. and were thawed ex tempore. Rabbits received a single slow intravenous bolus injection of test compound or vehicle alone in the marginal ear vein. Blood was sampled from the central ear artery. Rats and mice were injected subcutaneously or intraperitoneally. Rat blood samples were obtained under anesthesia (Forene) from the vena femoralis by puncture after incision of the skin. The mice were bled by orbita puncture after induction of anesthesia with pentobarbital. After clotting, blood samples were centrifuged (3000×g, 10 min) and the resulting sera were stored at −80° C. until assayed for DPP IV activity.

The above Examples show that all compounds tested were irreversible inhibitors. A good correlation was observed between the electron-withdrawing properties of the substituents and the inhibition of DPP IV. The methoxycarbonyl and alkylaminocarbonyl substituted derivatives were the most potent inhibitors with IC₅₀ values around 20 nM and inactivation rate constants around 3000 M⁻¹ s⁻¹. The same correlation was also observed between the electron-donating properties of the substituents and the stability in plasma. The most potent inhibitors are also the most unstable compounds. A notable exception is the good stability of the 4-acetylaminophenyl phosphonate ester (11e, t_{½}=320 min), together with a higher potency than could be expected (IC₅₀=0.4 μM, k=1900 M⁻¹ s⁻¹). Therefore, this compound together with the very potent 11n were further investigated in vitro and in vivo. These inhibitors showed no cytotoxicity in human peripheral blood mononuclear cells in concentrations up to 100 μM. The IC₅₀ values of 11e and 11n for inhibition of plasma DPP IV in rabbits upon single intravenous injection were around 0.2 mg/kg and below 20 μg/kg respectively. The compounds also showed no acute systemic or local toxicity, as was observed with the unsubstituted compound 5.

Due to the higher stability of 11e compared to 11n, it is believed that di(4-acetamidophenyl) 1-(S)-prolylpyrrolidine-2-(R,S)-phosphonate (11e) is a major improvement. The advantage of this compound compared to the pyrrolidine-2-nitrile reversible inhibitors is its long-lasting irreversible inhibition. Moreover, it is more stable than the frequently used boronic acid inhibitors.

1. A compound having a modulating activity on serine proteases and having the general formula 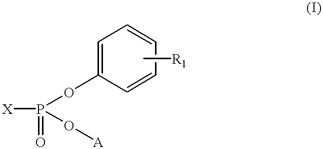
wherein
A is —R2 or H or C₁-C₆ alkyl or halogenoalkyl, except perfluoroalkyl;
the phenyl group is mono-, di- or trisubstituted with R1 or R2;
X is a peptide- or amino acid-derived moiety;
A and the phenyl group substituted with R1 may optionally form a biphenyl diester;
all R1 substituents and R2 substituents are each independently selected from the group consisting of:
a) C₁-C₆ acylamino;
b) aroylamino, optionally substituted at the o- and/or p- and/or m-position with alkyl, in particular C₁-C₆ alkyl, and/or a halogen;
c) C₁-C₆ alkylsulfonylamino;
d) arylsulfonylamino, optionally substituted at the o- and/or p- and/or m-position with alkyl, in particular C₁-C₆ alkyl, and/or a halogen;
e) α-aminoacylamino wherein the α-aminoacyl represents a side chain blocked or unblocked α-amino acid residue with the L, D or DL configuration at the α-carbon atom selected from the group consisting of: alanine, methionine, methionine sulfoxide, arginine, homoarginine, phenylalanine, aspartic acid, proline, hydroxyproline, asparagine, serine, cysteine, threonine, histidine, glycine, tyrosine, glutamic acid, pyroglutamic acid, tryptophan, glutamine, valine, norvaline, isoleucine, lysine, leucine, norleucine, thioproline, homoproline, 1,2,3,4-tetrahydroisoquinoline-3-carboxylic acid (Tic), 2,3-dihydroindol-2-carboxylic acid, α-naphtylglycine, α-phenylglycine, 4-amidinophenylglycine, 4-phenylproline, 4-amidinophenylalanine, O-benzyl tyrosine, omepa-acetyl lysine, α-aminobutyric acid, citrulline, homocitrilline, omithine, O-methylserine, O-ethylserine, S-methylcysteine, S-ethylcysteine, S-benzylcysteine, homoserine, 4-dehydroproline, penicillamine β-(2-thienyl)alanine, NH₂—CH(CH₂CHEt₂)—COOH, α-aminoheptanoic acid, NH₂—CH(CH₂-1-naphthyl)—COOH, NH₂—CH(CH₂-2-naphthyl)—COOH, NH₂—CH(CH₂-cyclohexyl)—COOH, NH₂—CH[CH—(cyclohexyl)₂]—COOH, NH₂—CH(CH₂-cyclopentyl)—COOH, NH₂—CH[CH—(cyclopentyl)₂]—COOH, NH₂—CH(CH₂-cyclobutyl)—COOH, NH₂—CH[CH-(cyclobutyl)₂]-COOH, NH₂—CH(CH₂-cyclopropyl-COOH, NH₂—CH[CH-(cyclopropyl)₂]-COOH, 5,5,5-trifluoroleucine, hexafluoroleucine, (S)-azetidine-2-carboxylic acid, (S)-pipecolic acid, (S)-oxazolidine-4-carboxylacid (L-thioproline), sarcosine;
f) residue selected from the group consisting of 3-aminobenzoic acid; ε-aminocaproic acid, β-alanine;
g) Y—NH—CO—NH—;
h) Y′O₂CCH(NHCO—Y)CH₂—;
i) Y′NHCO—;
j) CH₃—O—CO—Y′—NH—CO—;
k) CH₃—CH₂—O—CO—Y′—NH—CO—;
l) H, halogen, NO₂, CN, OH, COOH;
m) amino, C₁-C₆ alkylamine, C₂-C₁₂ dialkylamino;
n) C₁-C₆ acyl;
o) C₁-C₆ alkoxy-CO—; and
p) C₁-C₆ alkyl-S—,
wherein Y is C₁-C₆ alkyl, aryl or H and Y′ is C₁-C₆ alkyl, and pharmaceutically acceptable salts thereof.

2. The compound of claim 1, wherein X is a moiety of the general formula (AA)ₚ—aa—, wherein:
p indicates that there may be 0, 1, 2, 3, 4 or 5 residues AA, which can be the same or different within one molecule;
AA and aa are α-amino carboxylic acids with in a position an optionally substituted C₁-C₆ alkyl or aryl or aralkylmoiety; and
pharmaceutically acceptable salts thereof.

3. The compound of claim 1, wherein X is a moiety of the general formula (AA)ₚ—aa—, wherein:
p indicates that there may be 0, 1, 2, 3, 4 or 5 residues AA, which can be the same or different within one molecule;
AA and aa are selected from the group consisting of: alanine, methionine, methionine sulfoxide, arginine, homoarginine, phenylalanine, aspartic acid, proline, hydroxyproline, asparagine, serine, cysteine, threonine, histidine, glycine, tyrosine, glutamic acid, pyroglutamic acid, trytophan, glutamine, valine, norvaline, isoleucine, lysine, leucine, norleucine, thioproline, homoproline, 1,2,3,4-tetrahydroisoquinoline-3-carboxylic acid (Tic), 2,3-dihydroindol-2-carboxylic acid, α-naphtylglycine, α-phenylglycine, 4-amidinophenylglycine, 4-phenylproline, 4-amidinophenylalanine, O-benzyl tyrosine, omega-acetyl lysine, α-aminobutyric acid, citrulline, homocitrulline, omithine, O-methylserine, O-ethylserine, S-methylcysteine, S-ethylcysteine, S-benzylcysteine, homoserine, 4-dehydroproline, penicillamine β-(2-thienyl)alanine, NH₂-CH(CH₂CHEt₂)—COOH, α-aminoheptanoic acid, NH₂—CH(CH₂-1-naphthyl)—COOH, NH₂—CH(CH₂-2-naphthyl)—COOH, NH₂—CH(CH₂-cyclohexyl)—COOH, NH₂-CH[CH-(cyclohexyl)₂]-COOH, NH₂-CH(CH₂-cyclopentyl)—COOH, NH₂-CH[CH-(cyclopentyl)₂]-COOH, NH₂-CH(CH₂-cyclobutyl)—COOH, NH₂-CH[CH-(cyclobutyl)₂]-COOH, NH₂-CH(CH₂-cyclopropyl)—COOH, NH₂-CH[CH-(cyclopropyl)₂]-COOH, 5,5,5-trifluoroleucine, hexafluoroleucine, (S)-azetidine-2-carboxylic acid, (S)-pipecolic acid, (S)-oxazolidine-4-carboxylic acid, (R)-thiazolidine-4-carboxylacid (L-thioproline), 3-aminobenzoic acid, sarcosine, ε-aminocaproic acid, β-alanine, wherein the alpha amino residue may be side chain blocked or unblocked and has the L, D, or DL configuration at the alpha carbon atom; and
pharmaceutically acceptable salts thereof.

4. The compound of claim 2, wherein AA is lysine or ornithine, and the amino side chain thereof is involved in an intramolecular covalent bond.

5. The compound of claim 2, wherein X is M—(AA)ₚ—aa—, wherein:
M is selected from:
a) the group consisting of optionally substituted —CONH₂, —CSNH₂, —SO₂NH₂, phenyl-SO₂—, phenyl-CH₂SO₂—, 2-furyl-acryloyl;
b) the group of protecting group consisting of: acetyl, adamantyloxycarbonyl, benzyloxycarbonyl, benzoyl, benzyl, t-butoxycarbonyl, t-butyl, 2,4-dinitrophenyl, formyl, fluorenylmethoxycarbonyl, 4-methoxybenzyl, tosyl, trifluoroacetyl, trityl, phthaloyl, phenylalkylcarbonyl, 2-indanylacetyl, 2-(1,2,3,4-tetrahydronaphtyl)acetyl, 4-(4-benzylphenoxy) alkyl; and
pharmaceutically acceptable salts thereof.

6. The compound of claim 2, wherein X represents (M—)AA-aa—, M may or may not be present and aa is proline.

7. The compound of claim 2, wherein X represents (M—)AA-aa—, wherein M may or may not be present, and wherein AA and aa are both proline.

8. The compound of claim 2, wherein aa is alanine and R1 and R2 are selected from the group consisting of:
l) H, halogen, NO₂, CN, OH, COOH;
m) amino, C₁-C₆ alkylamine, C₂-C₁₂ dialkylamino;
n) C₁-C₆ acyl;
o) C₁-C₆ alkoxy-CO—;
p) C₁-C₆ alkyl-S—; and
pharmaceutically acceptable salts thereof.

9. The compound of claim 2, wherein at least the AA coupled to the aa is proline or phenylalanine and pharmaceutically acceptable salts thereof.

10. The compound of claim 9, which compound is Phe—Ala—diphenylphosphonate or Pro—Ala—diphenylphosphonate and pharmaceutically acceptable salts thereof.

11. The compound of claim 1, having the general formula: 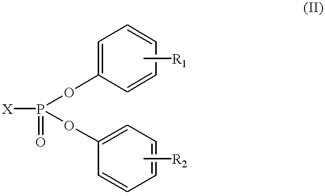
and pharmaceutically acceptable salts thereof.

12. The compound of claim 11, selected from the group consisting of:
Di(3 -acetamidophenyl) 1-(benzyloxycarbonyl-(S)-prolyl)pyrrolidine-2(R,S)-phosphonate;
Di(4-acetamidophenyl) 1-(benzyloxycarbonyl-(S)-prolyl)pyrrolidine-2(R,S)-phosphonate;
Di(4-methylsulfonylaminophenyl) 1-(benzyloxycarbonyl-(S)-prolyl)pyrrolidine-2(R,S)-phosphonate;
Di(3-ureylphenyl) 1-(benzyloxycarbonyl-(S)-prolyl)pyrrolidine-2(R,S)-phosphonate;
Di[4-(N-benzoylglycylamino)phenyl]-1-(benzyl-oxycarbonyl-(S)-prolyl)pyrrolidine-2(R,S)-phosphonate;
Di[4-(N-glycylamino)phenyl]-1-(benzyloxycarbonyl-(S)-prolyl)pyrrolidine-2(R,S)-phosphonate;
Di[4-(N-benzyloxycarbonyl-(S)-alanylamino)phenyl]-1-(benzyloxycarbonyl-(S)-prolyl)pyrrolidine-2(R,S)-phosphonate;
Di[4-((S)-pyroglutamylamino)phenyl]-1-(benzyloxycarbonyl-(S)-prolyl)pyrrolidine-2(R,S)-phosphonate;
Di{4-[-(S)-(2-methoxycarbonyl-2-acetamido)ethyl]-phenyl}1-(benzyloxycarbonyl-(S)-prolyl)pyrrolidine-2(R,S)-phosphonate;
Di(4-methoxycarbonylphenyl) 1-(tert-butyloxycarbonyl-(S)-prolyl)pyrrolidine-2(R,S)-phosphonate;
Di{4-[(ethoxycarbonyl)methylaminocarbonyl]phenyl}1-(benzyloxycarbonyl-(S)-prolyl)-pyrrolidine-2(R,S)-phosphonate;
Di{4-[2-(methoxycarbonyl)ethylaminocarbonyl]phenyl}1-(benzyloxycarbonyl-(S)-prolyl)-pyrrolidine-2(R,S)-phosphonate;
Di[4-(n-propylaminocarbonyl)phenyl]1-(benzyloxycarbonyl-(S)prolyl)-pyrrolidine-2(R,S)-phosphonate;
Di(3-acetamidophenyl) 1-((S)-prolyl)pyrrolidine-2(R,S)-phosphonate hydrochloride;
Di(4-acetamidophenyl) 1-((S)-prolyl)pyrrolidine-2(R,S)-phosphonate hydrochloride;
Di(4-methylsulfonylaminophenyl) 1-((S)-prolyl)-pyrrolidine-2(R,S)-phosphonate hydrochloride;
Di(3-ureylphenyl) 1-((S)-prolyl)pyrrolidine-2(R,S)-phosphonate hydrochloride;
Di[4-(N-benzoylglycylamino)phenyl]-1-((S)-prolyl)-pyrrolidine-2(R,S)-phosphonate hydrochloride;
Di[4-(N-glycylamino)phenyl]-1-((S)-prolyl)-pyrrolidine-2(R,S)-phosphonate trihydrochloride;
Di(4-(S)-alanylaminophenyl)-1-((S)-prolyl)pyrrolidine-2(R,S)-phosphonate trihydrochloride;
Di(4-(S)-pyroglutamylaminophenyl)-1-((S)-prolyl)-pyrrolidine-2(R,S)-phosphonate hydrochloride;
Di{4-[-(S)-(2-methoxycarbonyl-2-acetamido)ethyl]-phenyl}1-((S)-prolyl)-pyrrolidine-2-phosphonate hydrochloride; Di{4-[(ethoxycarbonyl)methylaminocarbonyl]phenyl}1-((S)-prolyl)pyrrolidine-2(R,S)-phosphonate;
Di{4-[2-(methoxycarbonyl)ethylaminocarbonyl]phenyl}1-((S)-prolyl)-pyrrolidine-2(R,S)-phosphonate hydrochloride;
Di[4-(n-propylaminocarbonyl)]1-((S)-prolyl)-pyrrolidine-2(R,S)-phosphonate hydrochloride; and
pharmaceutically acceptable salts thereof.

13. The compound of claim 1, wherein the compounds are 2,2′biphenyl diesters of α-aminoalkyl phosphonic acid having the general formula: 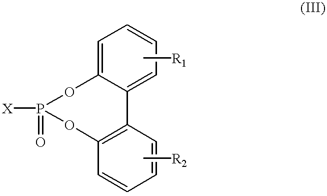
and pharmaceutically acceptable salts thereof.

14. The compound of claim 13, selected from the group consisting of:
2,2′-Biphenyl 1-(benzyloxycarbonyl-(S)-prolyl)-pyrrolidine-2(R,S)-phosphonate;
2,2′-Biphenyl 1-(t-butyloxycarbonyl-(S)-prolyl)-pyrrolidine-2(R,S)-phosphonate; and
2,2′-Biphenyl 1-((S)-prolyl)pyrrolidine-2(R,S)-phosphonate hydrochloride.

15. The compound of claim 14, which compound is 2,2′-Biphenyl 1-((S)-prolyl)pyrrolidine-2(R,S)-phosphonate hydrochloride or a pharmaceutically acceptable salt thereof.

16. The compound of claim 2, having the general formula: 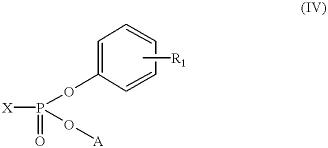
wherein A is H, C₁-C₆ alkyl or halogenoalkyl, except perfluoroalkyl, or pharmaceutically acceptable salts thereof.

17. The compound of claim 16, which compound is 2-(2′-Hydroxyphenyl)phenyl methyl 1-(S)-prolyl)-pyrrolidine-2(R,S)-phosphonate hydrochloride.

18. The compound of claim 1, wherein R1 and/or R2 is selected from the group consisting of 3—AcNH, 4—AcNH, 4—MeSO₂NH, 4—(N—Bz—Gly—NH), 4—(H—(S)—Ala—NH, 4—[(2S)—MeO₂CCH(NHAc)CH₂], 4—(S)—Pyr—NH, 4—(EtO₂CCH₂NHCO), 4—[MeO₂C(CH₂)₂NHCO], 4—[CH₃(CH₂)₂NHCO].

19. The compound of claim 5, wherein R1 and/or R is selected from the group consisting of 3—AcNH, 4—AcNH, 4—MeSO₂NH, 4—(N—Bz—Gly—NH), 4—(H—(S)—Ala—NH, 4—[(2S)—MeO₂CCH(NHAc)CH₂], 4—(S)—Pyr—NH, 4—(EtO₂CCH₂NHCO), 4—[MeO₂C(CH₂)₂NHCO], 4—[CH₃(CH₂)₂NHCO].

20. A pharmaceutical preparation comprising one or more of the compounds claimed in claim 1 and a suitable excipient, carrier or diluent.

21. The pharmaceutical preparation of claim 20, further comprising one or more additional therapeutic ingredients.

22. A method for in vitro modulating protease activity comprising the step of adding to a solution containing a peptide a concentration of the compound of claim 1 effective to modulate protease activity in the solution.

23. The method of claim 22, wherein the protease activity results in the unwanted degradation of a peptide substrate prior to measurement of the peptide substrate in a peptide assay.

24. A method for ex vivo inhibiting protease activity comprising the step of adding to a medium containing cells or organs a concentration of the compound of claim 1 effective to modulate protease activity in the medium.

25. The method of claim 24, wherein the cells or organs are for transplantation.

26. A method for in vivo modulating protease activity comprising the step of administering to a living organism an amount of the compound of claim 1 effective to modulate serum protease activity in the organism.
